SEQUENCE LISTING (22 sequences)

SEQ ID NO: 1 - DNA, 20 bp, synthetic construct
  source 1..20: mol_type = other DNA
tattggattgccacacggct

SEQ ID NO: 2 - DNA, 20 bp, synthetic construct
  source 1..20: mol_type = other DNA
aaagcgacccaaaggtggat

SEQ ID NO: 3 - DNA, 20 bp, synthetic construct
  source 1..20: mol_type = other DNA
tttctgttacggtccagggc

SEQ ID NO: 4 - DNA, 21 bp, synthetic construct
  source 1..21: mol_type = other DNA
acacatccagtttgctaggct

SEQ ID NO: 5 - DNA, 19 bp, synthetic construct
  source 1..19: mol_type = other DNA
tggggaagacaaccaggac

SEQ ID NO: 6 - DNA, 18 bp, synthetic construct
  source 1..18: mol_type = other DNA
ttcggcaatagcaggtgg

SEQ ID NO: 7 - DNA, 23 bp, synthetic construct
  source 1..23: mol_type = other DNA
gaggatgcaagctcaagtcttag

SEQ ID NO: 8 - DNA, 19 bp, synthetic construct
  source 1..19: mol_type = other DNA
tgcaatggcaagtgaaagc

SEQ ID NO: 9 - DNA, 22 bp, synthetic construct
  source 1..22: mol_type = other DNA
actgctatgccttgtttttgtc

SEQ ID NO: 10 - DNA, 22 bp, synthetic construct
  source 1..22: mol_type = other DNA
ctactccactcccaaccttctc

SEQ ID NO: 11 - DNA, 22 bp, synthetic construct
  source 1..22: mol_type = other DNA
cgcactcagttatggcttctac

SEQ ID NO: 12 - DNA, 18 bp, synthetic construct
  source 1..18: mol_type = other DNA
gtgattctgctccccgag

SEQ ID NO: 13 - DNA, 21 bp, synthetic construct
  source 1..21: mol_type = other DNA
ctccttttcttccctgcctag

SEQ ID NO: 14 - DNA, 23 bp, synthetic construct
  source 1..23: mol_type = other DNA
tctggtagacaacaccattcttg

SEQ ID NO: 15 - DNA, 19 bp, synthetic construct
  source 1..19: mol_type = other DNA
ccctcatcagcgaacagtg

SEQ ID NO: 16 - DNA, 22 bp, synthetic construct
  source 1..22: mol_type = other DNA
caggatgtcattgtccagagtc

SEQ ID NO: 17 - DNA, 18 bp, synthetic construct
  source 1..18: mol_type = other DNA
cggcgttctctcaggtga

SEQ ID NO: 18 - DNA, 19 bp, synthetic construct
  source 1..19: mol_type = other DNA
tggcattgtccagtgcttc

SEQ ID NO: 19 - DNA, 21 bp, synthetic construct
  source 1..21: mol_type = other DNA
tgctactgacaacgtggcttc

SEQ ID NO: 20 - DNA, 20 bp, synthetic construct
  source 1..20: mol_type = other DNA
acatctgaggctccgacatc

SEQ ID NO: 21 - DNA, 20 bp, synthetic construct
  source 1..20: mol_type = other DNA
gtggagaacatgctggacct

SEQ ID NO: 22 - DNA, 20 bp, synthetic construct
  source 1..20: mol_type = other DNA
gccaatgggactggtaattc

(19) United States
(12) Patent Application Publication
(10) Pub. No.: US 2026/0209712 A1
(43) Pub. Date: Jul. 23, 2026

(54) METHODOLOGIES TO GENERATE HUMAN PANETH CELLS AND ENTEROCHROMAFFIN CELLS AND DETERMINE THEIR RESPONSES

(71) Applicant: Cedars-Sinai Medical Center, Los Angeles, CA (US)

(72) Inventors: Robert Barrett, Los Angeles, CA (US); Stephan Targan, Santa Monica, CA (US); Shachi Patel, Sherman Oaks, CA (US); Monica Wagner, Los Angeles, CA (US)

(73) Assignee: Cedars-Sinai Medical Center, Los Angeles, CA (US)

(21) Appl. No.: 19/458,165

(22) Filed: Jan. 23, 2026

Related U.S. Application Data
(60) Provisional application No. 63/748,768, filed on Jan. 23, 2025.

(51) Int. Cl. C12N 5/071

(52) U.S. Cl. CPC C12N 5/0679; C12N 2500/32; C12N 2501/11; C12N 2501/119; C12N 2501/155; C12N 2501/16; C12N 2501/415; C12N 2501/727; C12N 2501/734; C12N 2501/999; C12N 2506/45; C12N 2509/00; C12N 2533/90

(57) ABSTRACT

Described herein are methods for enrichment of Paneth cells, as well as enterochromaffin cells, goblet cells or both in iPSC-derived human intestinal organoids. Also described are methods of determining the responses of these aforementioned cells.

### CROSS-REFERENCE TO RELATED APPLICATIONS

This application includes a claim of priority under 35 U.S.C. § 119(e) to U.S. provisional patent application No. 63/748,768, filed Jan. 23, 2025, the entirety of which is hereby incorporated by reference.

### STATEMENT REGARDING FEDERALLY SPONSORED RESEARCH OR DEVELOPMENT

This invention was made with Government support under Grant No. DK123511 awarded by National Institutes of Health. The Government has certain rights in the invention.

### REFERENCE TO SEQUENCE LISTING

This application contains a Sequence Listing submitted as a computer readable form named “SeqListing_065472_001008USPT.xml”, having a size in bytes of 20,498 bytes, and created on Jan. 23, 2026. The information contained in this computer readable form is hereby incorporated by reference in its entirety.

### FIELD OF INVENTION

This invention relates to differentiation and enrichment of Paneth cells, enterochromaffin cells, and goblet cells, as well as determining their responses to various stimuli.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) is an inflammatory GI disorder which affects 3 million individuals in the US. It is caused by a dysregulated immune response to the intestinal microbiome in genetically susceptible individuals. Paneth cells secrete antimicrobial peptides which modulate the microbiome. There is substantial evidence that Paneth cells play a role in CD but it is exceptionally difficult to determine their role in this.

The problem is, currently, there is no meaningful in vitro way to study Paneth cells. Paneth cells are highly reactive to their stimuli so animal models are challenging as Paneth cells may react strongly to host cytokines or their microbiome and thus mechanistic studies, such as determining the role of specific cytokines or microbes are challenging. Attempts to culture intestinal tissue ex vivo have failed to maintain a Paneth cell population. One study found there was an enriched population of Paneth cell in human biopsy derived organoids (He et al, Cell Stem Cell, 2023) but organoids contained only 1% Paneth cells. However there were very limited functional analyses. Some have used murine models but the human correlation is doubtful and the confounding influence of host microbiome, genetics and other environmental triggers are difficult to assess. Some have attempted to use freshly resected intestinal cells but these die quickly.

According, there remains a need in the art for methods of generating meaningful numbers of Paneth cells.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of differentiating epithelial human intestinal organoids into Paneth Cells and enriching the Paneth Cells in eHIO culture, comprising:
seeding about 25,000-75,000 human epithelial intestinal cells into a MATRIGEL bubble and maintaining the human epithelial intestinal cells in eHIO expansion medium for about 1-3 days;
replacing the eHIO medium with differentiation medium and culturing for about 4-6 days;
replating the human epithelial intestinal cells and continuing to culture in differentiation medium for an additional 4-6 days thereby generating a cell culture enriched with Paneth cells; and
harvesting cells from the cell culture enriched with Paneth cells.

In various embodiments the method can comprise:
seeding about 35,000-65,000 human epithelial intestinal cells into a MATRIGEL bubble and maintaining the human epithelial intestinal cells in eHIO medium for about 1.5-2.5 days;
replacing the eHIO medium with differentiation medium and culturing for about 4.5-5.5 days;
replating the human epithelial intestinal cells and continuing to culture in differentiation medium for an additional 4.5-5.5 days thereby generating a cell culture enriched with Paneth cells; and
harvesting cells from the cell culture enriched with Paneth cells.

In various embodiments the method can comprise:
seeding about 50,000 human epithelial intestinal cells into a Matrigel bubble and maintaining the human epithelial intestinal cells in eHIO expansion medium for about 2 days;
replacing the eHIO expansion medium with differentiation medium and culturing for about 5 days;
replating the human epithelial intestinal cells and continuing culture in differentiation medium for an additional 5 days, thereby generating a cell culture enriched with Paneth cells; and
harvesting cells from the cell culture enriched with Paneth cells.

In various embodiments, the method can further comprise analyzing Paneth cell numbers and gene expression and/or protein expression using flow cytometry, qPCR, immunocytochemistry, or combinations thereof.

In various embodiments, the Paneth cells can have an increased mRNA expression of one or more genes selected from the group consisting of DEFA5, DEFA6, PLA2G2A, REG3A, and ITLN2, as compared to undifferentiated eHIOs. In various embodiments, the Paneth cells can have an increased mRNA expression of DEFA5, DEFA6, PLA2G2A, and REG3A, as compared to undifferentiated eHIOs. In various embodiments, the Paneth cells can have an increased mRNA expression of HD5, HD6, PLA2AG2A, and REG3A. In various embodiments, the Paneth cells can have a presence of lysozyme, HD5, REG3A, or both, as compared to undifferentiated eHIOs. In various embodiments, the cell culture comprising Paneth cells can show a presence of electron dense granules via immunocytochemistry. In various embodiments, the Paneth cells can be LYZ⁺ and DEFA5⁺.

Various embodiments of the invention provide for a method of differentiating epithelial human intestinal organoids into enterochromaffin cells, goblet cells or both and enriching the enterochromaffin cells, goblet cells, or both in eHIO culture, comprising:
seeding about 25,000-75,000 human epithelial intestinal cells into a MATRIGEL bubble and maintaining the human epithelial intestinal cells in eHIO medium for about 1-3 days;
replacing the eHIO medium with differentiation medium and culturing for about 4-6 days;
replating the human epithelial intestinal cells and continuing to culture in differentiation medium for an additional 4-6 days thereby generating a cell culture enriched with enterochromaffin cells, goblet cells, or both; and
harvesting cells from the cell culture enriched with enterochromaffin cells, goblet cells, or both.

In various embodiments, the method can comprise:
seeding about 35,000-65,000 human epithelial intestinal cells into a MATRIGEL bubble and maintaining the human epithelial intestinal cells in eHIO medium for about 1.5-2.5 days;
replacing the eHIO medium with differentiation medium and culturing for about 4.5-5.5 days;
replating the human epithelial intestinal cells and continuing to culture in differentiation medium for an additional 4.5-5.5 days thereby generating a cell culture enriched with enterochromaffin cells, goblet cells, or both; and
harvesting cells from the cell culture enriched with enterochromaffin cells, goblet cells, or both.

In various embodiments, the method can comprise:
seeding about 50,000 human epithelial intestinal cells into a Matrigel bubble and maintaining the human epithelial intestinal cells in eHIO medium for about 2 days;
replacing the eHIO medium with differentiation medium and culturing for about 5 days;
replating the human epithelial intestinal cells and continuing culture in differentiation medium for an additional 5 days thereby generating a cell culture enriched with enterochromaffin cells, goblet cells, or both; and
harvesting cells from the cell culture enriched with enterochromaffin cells, goblet cells, or both.

In various embodiments, the method can further comprise analyzing enterochromaffin cell numbers, goblet cell numbers, or both and gene expression and/or protein expression using flow cytometry, qPCR, immunocytochemistry, or combinations thereof.

In various embodiments, the enterochromaffin cells, goblet cells, or both can have increased mRNA expression of CHGA and MUC2, as compared to undifferentiated eHIOs. In various embodiments, the enterochromaffin cells can be CHGA⁺ and serotonin⁺.

In various embodiments, the eHIO medium can comprise Adv DMEM/F12, L-glut, B27, EGF, Noggin, CHIR, SB202190, A8301, and optionally, Rock inhibitor, and Penicillin-Streptomycin. In various embodiments, the CHIR can be at a concentration of at least 3 mM.

In various embodiments, the differentiation medium can comprise Adv DMEM/F12, Penicillin-Streptomycin, 200 mM L-glut, B27, EGF, Noggin, CHIR, and DAPT. In various embodiments, the CHIR can be at a concentration of at least 3 mM. In various embodiments, the differentiation medium can further comprise IL-22.

In various embodiments, the human epithelial intestinal cells can be differentiated from human intestinal organoids (HIOs) that have been cultured for about 15-50 days before being sorted and grown as epithelial only-human intestinal organoids. In various embodiments, the human epithelial intestinal cells can be differentiated from human intestinal organoids (HIOs) that have been cultured for about 25-35 days before being sorted and grown as epithelial only-human intestinal organoids.

In various embodiments, the human epithelial intestinal cells can be differentiated from human intestinal organoids (HIOs) that have been cultured for at least 30 days before being sorted and grown as epithelial only-human intestinal organoids. In various embodiments, the human epithelial intestinal cells can be differentiated from human intestinal organoids (HIOs) that have been cultured for about 30 days before being sorted and grown as epithelial only-human intestinal organoids.

In various embodiments, the human epithelial intestinal cells can be differentiated from human intestinal organoids (HIOs) that have been passaged for at least 5 times. In various embodiments, the human epithelial intestinal cells can be differentiated from human intestinal organoids (HIOs) that have been passaged for at least 10 times. In various embodiments, the human epithelial intestinal cells can be differentiated from human intestinal organoids (HIOs) that have been passaged for at least 15 times. In various embodiments, the human epithelial intestinal cells can be differentiated from human intestinal organoids (HIOs) that have been passaged for about 5-30 times. In various embodiments, the human epithelial intestinal cells can be differentiated from human intestinal organoids (HIOs) that have been passaged for about 10-15 times.

In various embodiments, the epithelial human intestinal cells can be obtained by:
removing HIOs from MATRIGEL and washing the HIOs;
dissociating the HIOs into dissociated cells;
separating and isolating EpCAM+ cells;
resuspending EpCAM+ cells in MATRIGEL as single cells, wherein the EpCAM+ cells self-organize and expand to form epithelial-only HIOs (eHIOs);
maintaining the eHIOs in organoid medium supplemented with SB202190 and A83-01; and
dissociating the eHIOs into single cell suspension.

In various embodiments, dissociating the HIOs into dissociated cells can comprise incubating the HIOs in cell detachment enzyme to dissociate the cells. In various embodiments, separating and isolating EpCAM+ cells can comprise incubating the dissociated cells with EpCAM MicroBeads.

Various embodiments of the invention provide for a method of incorporating enriched human Paneth cell organoids in a transwell culture, comprising:
dissociating epithelial human intestinal organoids (eHIOs) enriched for human Paneth cells by:
   removing a MATRIGEL bubble off a plate comprising eHIOs enriched for human Paneth cells;
   breaking up the MATRIGEL bubble with medium and transferring the mixture into a tube;
   centrifuging at about 1000-2000 rpm for about 1-5 minutes thereby generating supernatant and pellet;
   aspirating the supernatant to remove the MATRIGEL;
   adding about 1-5 mL PBS to the pellet, breaking organoids out of the MATRIGEL, and repeating 1-3 times;
   centrifuging at about 1000-2000 rpm for about 1-5 minutes thereby generating supernatant and pellet;
   removing the supernatant to remove MATRIGEL;
   resuspending the pellet in about 0.5-1.5 mL TrypLE Select;
   incubating at about 34-40° C. for about 10-14 minutes;
   neutralizing with about 1-3 mL of about 12-14% FBS/PBS solution;
   centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, the method can comprise:
dissociating epithelial human intestinal organoids (eHIOs) enriched for human Paneth cells by:
   scraping a MATRIGEL bubble off a plate comprising eHIOs enriched for human Paneth cells;
   breaking up the MATRIGEL bubble with medium and transferring the mixture into a 15 mL conical tube;
   centrifuging at about 1000-2000 rpm for about 1-5 minutes thereby generating supernatant and pellet;
   aspirating the supernatant to remove MATRIGEL;
   adding about 1-5 mL PBS to the pellet, breaking organoids out of the MATRIGEL, and repeating 1-3 times;
   centrifuging at about 1000-2000 rpm for about 1-5 minutes thereby generating supernatant and pellet;
   removing the supernatant to remove MATRIGEL;
   resuspending the pellet in about 0.5-1.5 mL TrypLE Select;
   incubating at about 34-40° C. for about 10-14 minutes;
   neutralizing with about 1-3 mL of about 12-14% FBS/PBS solution;
   centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, the method can comprise:
dissociating eHIOs enriched for human Paneth cells by:
   using a P1000 pipette or a 5 mL serological pipette scrape a MATRIGEL bubble off a plate comprising eHIOs enriched for human Paneth cells;
   triturating the MATRIGEL bubble with medium and transferring the mixture into a 15 ml conical tube;
   centrifuging at about 1500 rpm for about 3 minutes thereby generating supernatant and pellet;
   aspirating the supernatant to remove MATRIGEL but not the organoids;
   adding about 3 mL PBS to the pellet, triturating to break organoids out of the MATRIGEL, and repeat the wash twice;
   centrifuging at about 1500 rpm for about 3 minutes thereby generating supernatant and pellet;
   aspirating the supernatant, ensuring only clean Matrigel is removed, leaving the organoids intact;
   resuspending the pellet in about 1 mL TrypLE Select;
   incubating at about 37° C. for about 12 minutes;
   neutralizing with about 2 mL of about 10% FBS/PBS solution;
   centrifuging at about 1500 rpm for about 3 minutes;
resuspending cells at a density of about 4×10⁶ cells/ml and seed about 4×10⁵ cells per transwell; and
culturing the cells for about 24 hrs.

Various embodiments of the invention provide for a method of incorporating enriched human into enterochromaffin cells, goblet cells or both in a transwell culture, comprising:
dissociating epithelial human intestinal organoids (eHIOs) enriched for enterochromaffin cells, goblet cells or both by:
   removing a MATRIGEL bubble off a plate comprising eHIOs enriched for human into enterochromaffin cells, goblet cells or both;
   breaking up the MATRIGEL bubble with medium and transferring the mixture into a tube;
   centrifuging at about 1000-2000 rpm for about 1-5 minutes thereby generating supernatant and pellet;
   aspirating the supernatant to remove the MATRIGEL;
   adding about 1-5 mL PBS to the pellet, breaking enterochromaffin cells, goblet cells or both out of the MATRIGEL, and repeating 1-3 times;
   centrifuging at about 1000-2000 rpm for about 1-5 minutes thereby generating supernatant and pellet;
   removing the supernatant to remove MATRIGEL;
   resuspending the pellet in about 0.5-1.5 mL TrypLE Select;
   incubating at about 34-40° C. for about 10-14 minutes;
   neutralizing with about 1-3 mL of about 12-14% FBS/PBS solution;
   centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, wherein the method can comprise:
dissociating epithelial human intestinal organoids (eHIOs) enriched for enterochromaffin cells, goblet cells or both by:
   scraping a MATRIGEL bubble off a plate comprising eHIOs enriched for human into enterochromaffin cells, goblet cells or both;
   breaking up the MATRIGEL bubble with medium and transferring the mixture into a 15 ml conical tube;
   centrifuging at about 1000-2000 rpm for about 1-5 minutes thereby generating supernatant and pellet;
   aspirating the supernatant to remove MATRIGEL;
   adding about 1-5 mL PBS to the pellet, breaking enterochromaffin cells, goblet cells or both out of the MATRIGEL, and repeating 1-3 times;
   centrifuging at about 1000-2000 rpm for about 1-5 minutes thereby generating supernatant and pellet;
   removing the supernatant to remove MATRIGEL;
   resuspending the pellet in about 0.5-1.5 mL TrypLE Select;
   incubating at about 34-40° C. for about 10-14 minutes;
   neutralizing with about 1-3 mL of about 12-14% FBS/PBS solution;
   centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, the method can comprise:
dissociating eHIOs enriched for human into enterochromaffin cells, goblet cells or both by:
   using a P1000 pipette or a 5 mL serological pipette scrape a MATRIGEL bubble off a plate comprising eHIOs enriched for human into enterochromaffin cells, goblet cells or both;
   triturating the MATRIGEL bubble with medium and transferring the mixture into a 15 ml conical tube;
   centrifuging at about 1500 rpm for about 3 minutes thereby generating supernatant and pellet;
   aspirating the supernatant to remove MATRIGEL but not the enterochromaffin cells, goblet cells or both;
   adding about 3 mL PBS to the pellet, triturating to break organoids out of the MATRIGEL, and repeat the wash twice;
   centrifuging at about 1500 rpm for about 3 minutes thereby generating supernatant and pellet;
   aspirating the supernatant, ensuring only clean Matrigel is removed, leaving the enterochromaffin cells, goblet cells or both intact;
   resuspending the pellet in about 1 mL TrypLE Select;
   incubating at about 37° C. for about 12 minutes;
   neutralizing with about 2 mL of about 10% FBS/PBS solution;
   centrifuging at about 1500 rpm for about 3 minutes;
resuspending cells at a density of about 4×10⁶ cells/ml and seed about 4×10⁵ cells per transwell; and culturing the cells for about 24 hrs.

In various embodiments, the method can further comprise treating the cells with cytokines, microbes, or microbial ligands, or combinations thereof for about 24-72 hrs. In various embodiments, the method can further comprise treating the cells with cytokines, microbes, or microbial ligands, or combinations thereof for about 48 hrs.

In various embodiments, the transwells can be coated with MATRIGEL for about 1-3 hrs at room temperature. In various embodiments, the transwells (about 0.33 cm²) can be coated with MATRIGEL for about 2 hrs at room temperature.

Various embodiments of the invention provide for human Paneth cell organoids obtained by any one of the methods of the invention as described herein.

Various embodiments of the invention provide for human Paneth cell organoid model, comprising: any one of the human Paneth cell organoids of the invention as described herein.

Various embodiments of the invention provide for a transwell culture, comprising: any one of the human Paneth cell organoid models of the invention as described herein.

Various embodiments of the invention provide for enterochromaffin cells, goblet cells or both obtained by any one of the methods of the invention as described herein.

Various embodiments of the invention provide for an enterochromaffin cells model, goblet cell model or both, comprising enterochromaffin cells, goblet cells or both, of the invention as described herein.

Various embodiments of the invention provide for a transwell culture obtained by any one of the methods of the invention as described herein.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1D depicts the characterization of an enriched Paneth population in iPSC-derived epithelial only-HIOs (eHIOs). 1A) Representative flow cytometry dot plots of lysozyme+ cells in eHIOs cultured in maintenance media (EGF (100 ng/ml), Noggin (100 ng/ml and CHIR99021 (2 mM), SB202190 (10 mM) and A8301 (500 nM) (ENC(2)+SA) and those treated with an increased concentration of CHIR99021 (3 mM); ENC(3)) either alone, or in combination with IL-22 (2 ng/ml; ENC(3)+IL22) or DAPT (10 mM; ENC(3)+D) with an accompanying graph showing data from 7 independent experiments. 1B) qRT-PCR of Paneth cell related genes in eHIOs cultured either in maintenance media or ENC(3)+D. Students t test *P<0.05, ***P<0.001, ****P<0.0001 as compared to maintenance media. Each value represents Mean±SEM. Data from 8 independent experiments. 1C) Representative fluorescent images showing eHIOs, cultured either in maintenance media or ENC(3)+D, immunopositive for E-cadherin (red) and lysozyme, DEFA5, REG3A, and ITLN2 (all green). Scale bar is 100 mm for upper two panel and 25 mm for lower panel. Lower panel are enlarged images of broken white rectangle in middle panel. 1D) Micrograph showing presence of electron dense granules (orange arrows) in eHIOs treated with ENC(3)+D. Scale bar is 6 mm.

FIG. 2 (panels A-H) depicts functional responses of iPSC-derived Paneth cells. A) Representative flow cytometry dot plots of lysozyme+ and DEFA5+ cells in eHIOs cultured with ENC(3)+D and treated either with/without IL22 (10 ng/ml) with an accompanying graph showing data from 7 independent experiments. B) qRT-PCR of Paneth cell related genes in ENC(3)+D treated eHIOs with/without IL-22 (10 ng/ml). Students t test *P<0.05, **P<0.01 as compared to no addition of IL-22. Data from 4 independent experiments. C) Representative fluorescent images showing enriched Paneth cell eHIOs are immunopositive for TLR2, TLR5, and NOD2 (all red), lysozyme (green) and E-cadherin (white). Scale bar is 50 mm. D) Secretion of lysozyme in Paneth cell enriched monolayers in response to apical administration of MDP, LPS, Pam3CSKJ4 and Flagellin. Undifferentiated monolayers were included as control (right panel). ***P<0.001 as compared to untreated control. Data from 3-4 independent experiments. E) Schematic of studies utilizing microbial cultures obtained from inflammatory bowel disease patients. F) Heatmap of differentially secreted proteins in media obtained from Paneth cell enriched monolayers collected following 30-minute exposure to bacterial enriched suspensions derived from Crohn's disease mucosal scrapings. Samples are grouped as Treated (bacteria-exposed) or Untreated (control). Data represent mean values from three independent experiments. G) Volcano plot of differential protein expression between Treated and Untreated secretome samples. Log₂ fold change (log 2FC) is plotted against −log₁₀ adjusted p-value (FDR). Proteins meeting significance thresholds (FDR<0.05, |log₂FC|>1) are labeled. Data represent combined results from three independent experiments. H) Gene Ontology (GO) Biological Process enrichment analysis of proteins significantly altered in the secretome following bacterial treatment. Terms are ranked by enrichment significance (−log₁₀ FDR). Circle size reflects the number of associated proteins, and color indicates FDR. Analysis performed using STRING (version 12.0) based on data from three independent experiments.

FIG. 3 (panels A-D) depicts the characterization of HIOs and eHIOs. A) Representative fluorescent images of Day10 and Day30 HIOs immunopositive for E-cadherin (red) and lysozyme, DEFA5, REG3A and ITLN2 (green). Scale bar=100 mm. B) Schematic illustrating the generation of eHIOs from iPSCs. C) Representative fluorescent images of Day10 and Day30 e-HIOs immunopositive for E-cadherin (red) and lysozyme, DEFA5, REG3A and ITLN2 (green). Scale bar=50 mm. D) qPCR analysis of CHGA and MUC2 expression in eHIOs cultured in ENC(2)+SA or ENC(3)+DAPT. Data represent mean±SEM from 3 biological replicates. Students t test: *P<0.05 **P<0.01 versus ENC(2)+SA.

FIG. 4 (panels A-D) depicts the characterization of an enriched Paneth cell population in iPSC-derived eHIOs from 2 additional control lines, along with characterization of Paneth cell-enriched in transwell cultures. A) Representative flow cytometry dot plots showing lysozyme⁺ and DEFA5⁺ cells in eHIOs generated from control lines 2GW3i and 9EWPi, cultured in ENC(2)+SA or ENC(3)+DAPT media with an accompanying quantification graph from 5 independent experiments. B) qRT-PCR analysis of Paneth cell-related genes (DEFA5, DEFA6, PLA2G2A, ITLN2, and REG3A) in eHIOs from each control line cultured in ENC(2)+SA or ENC(3)+DAPT. C) qRT-PCR characterization of Paneth cell markers (DEFA5, DEFA6, PLA2G2A, ITLN2, REG3A) in Paneth cell-enriched monolayers seeded on Transwell inserts and cultured in ENC(2)+SA or ENC(3)+DAPT. D) qRT-PCR analysis of enteroendocrine (CHGA) and goblet cell (MUC2) markers in Paneth cell-enriched transwell cultures maintained in ENC(2)+SA or ENC(3)+DAPT. Each value represents Mean±SEM. Students t test *P<0.05, **P<0.01 and ***P<0.001 as compared to ENC(2)+SA media.

FIG. 5 shows expression of NOD2, TLR2 and TLR5 in Paneth cell related genes in untreated or treated eHIOs. Representative images from 3 individual experiments. E-cadherin (red) counterstained with selected microbial sensor (green). All images (×20).

FIG. 6 shows flow cytometry analysis of treated eHIOs. Representative dot plots and graphical representation of 4 individual experiments.

FIG. 7 shows Expression of Paneth cell related genes in untreated treated eHIOs. qPCR of 4 individual experiments comparing eHIOs cultured in proliferation media as compared those cultured in CHIR99021 and DAPT. *p<0.05, **p<0.01, ***p<0.001, and ****p<0.0001 as compared to gene expression in proliferation media.

FIG. 8 shows immunocytochemistry of antimicrobial peptides in untreated or treated eHIOs. Representative images from 2 individual experiments comparing eHIOs cultured in proliferation media as compared those cultured in CHIR99021 and DAPT. E-cadherin (red) counterstained with selected antimicrobial peptide (green). All images (×20).

FIG. 9A-9B shows the characterization of enteroendocrine cell populations in epithelial-only iPSC-derived intestinal organoids. (9A) Representative flow cytometry analysis of CHGA⁺ and serotonin⁺ cells in epithelial-only intestinal organoids derived from two independent iPSC lines (03i and 9EWPi), with corresponding quantification. (9B) Representative immunocytochemistry images of undifferentiated and differentiated epithelial-only intestinal organoids showing expression of CHGA, serotonin, and DDC.

FIG. 10 (panels A-B) show transcriptional profiling of enteroendocrine differentiation in epithelial-only iPSC-derived intestinal organoids. qPCR analysis of enteroendocrine-associated gene expression in epithelial-only intestinal organoids derived from the 03i (A) and 9EWPi (B) iPSC lines.

FIG. 11 (panels A-F) show Single-cell transcriptional profiling of epithelial cell populations in iPSC-derived organoids. (A-B) UMAP projections of single cells colored by differentiation state and by sample, showing transcriptional separation between conditions. C) UMAP projection colored by Leiden clustering, identifying transcriptionally distinct cell clusters. D) UMAP projection annotated by cell type assignment based on majority-voting, identifying intestinal epithelial populations including enteroendocrine, goblet, tuft, transit-amplifying, and differentiated epithelial cells. E) Dot plot showing expression of enteroendocrine-associated genes across clusters, with dot size indicating the fraction of cells expressing each gene and color indicating mean expression. F) Dot plot showing expression of marker genes defining enteroendocrine cell subtypes across annotated enteroendocrine populations.

FIG. 12 shows serotonin secretion by epithelial-only iPSC-derived intestinal organoids. Fold change in serotonin secretion relative to untreated controls measured in culture supernatants from epithelial-only intestinal organoids following treatment with the indicated compounds, as determined by ELISA.

FIG. 13 (panels A-C) shows the characterization of goblet cells in epithelial-only iPSC-derived intestinal organoids. (A) Representative flow cytometry analysis of MUC2⁺ goblet cells in epithelial-only intestinal organoids derived from two independent iPSC lines (03i and 9EWPi), with corresponding quantification. (B) qPCR analysis of MUC2 expression in epithelial-only intestinal organoids derived from the 03i and 9EWPi iPSC lines. (C) Representative immunocytochemistry images showing MUC2 expression in epithelial-only intestinal organoids.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Paneth cells are a specialized intestinal epithelial subtype, which reside in the crypt of the small intestine, and whose major function is to produce antimicrobial peptides (AMPs). Genetic variants in the NOD2 and ATG16L1 genes are associated with an altered Paneth cell phenotype but the confounding influences of the microbiome and mucosal immune system make it challenging to determine whether genetic variations or environmental influences are the cause of these altered phenotypes. Therefore, our goal was to develop a biologically responsive in vitro Paneth cell model that would permit an examination of both intrinsic and extrinsic influences in a controlled reductionist system.

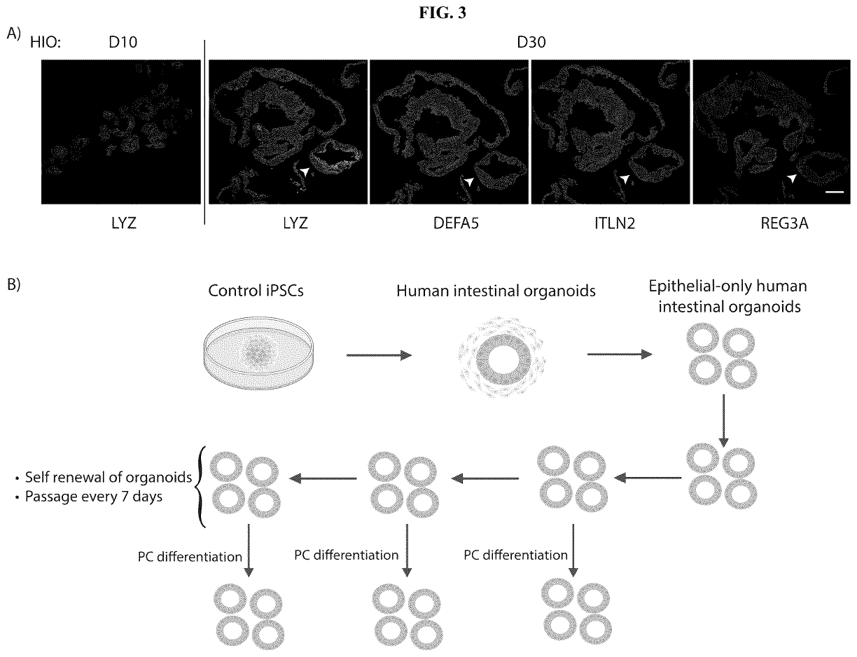

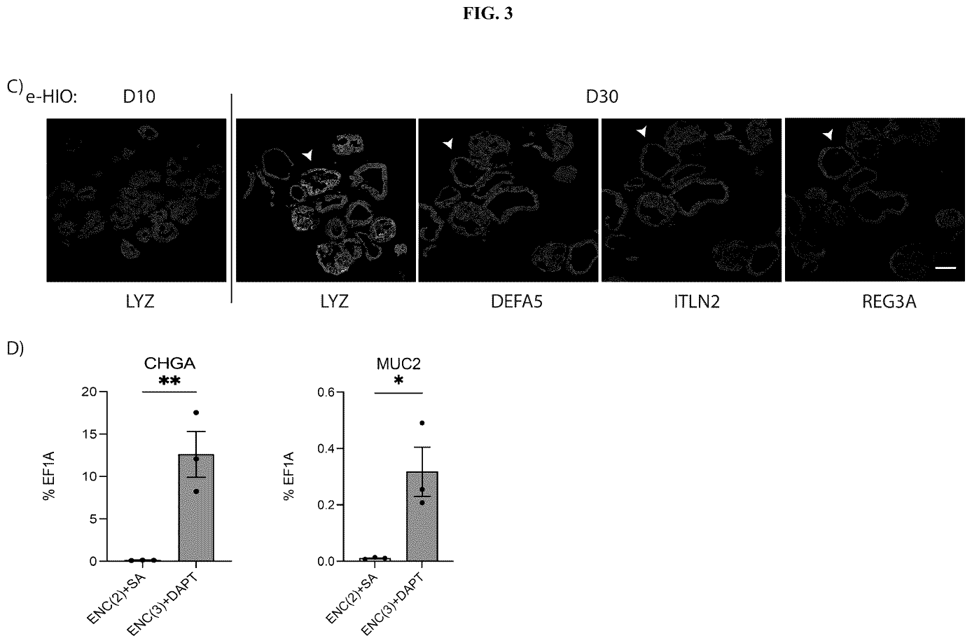

We utilized induced pluripotent stem cells (iPSCs) for the source of our human intestinal organoids (HIOs), as this is a donor cell type that can be obtained either from a simple blood draw or lymphoblastoid cell lines from numerous well characterized biorepositories and iPSC-derived HIOs have previously been shown to representative of the small intestine. We directed a control iPSC line (CS03iCTR-n1) to HIOs and the initial characterization of our 10d iPSC-derived HIOs revealed a complete absence of lysozyme+ cells. After 30d, only 2 organoids in one series of experiments were found to be lysozyme+ but were negative for additional AMPs (FIG. 3A). Given the near absence of Paneth cells, we thus aimed to enrich our organoids for this cell type. Numerous attempts have been made to enrich for Paneth cells in various modalities such as murine organoids and human biopsy-derived intestinal organoids and so to test a subset of these approaches, we first purified the epithelial cellular component of 10d and 30d HIOs, cultured them as epithelial-only HIOs (eHIOs), which could be serially passaged every 7 days, with the goal of examining such approaches (FIG. 3B). Given that we found that eHIOs from d10 iPSC-derived-HIOs again had no lysozyme+ cells while those from d30 iPSC-derived HIOs contained a small presence (FIG. 3C), we thus utilized eHIOs from the d30 timepoint in the studied discussed herein.

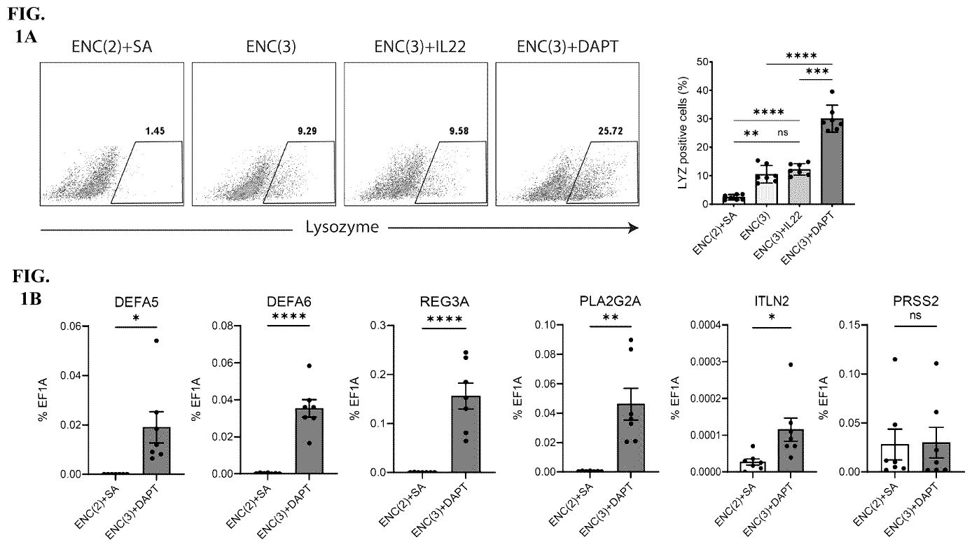

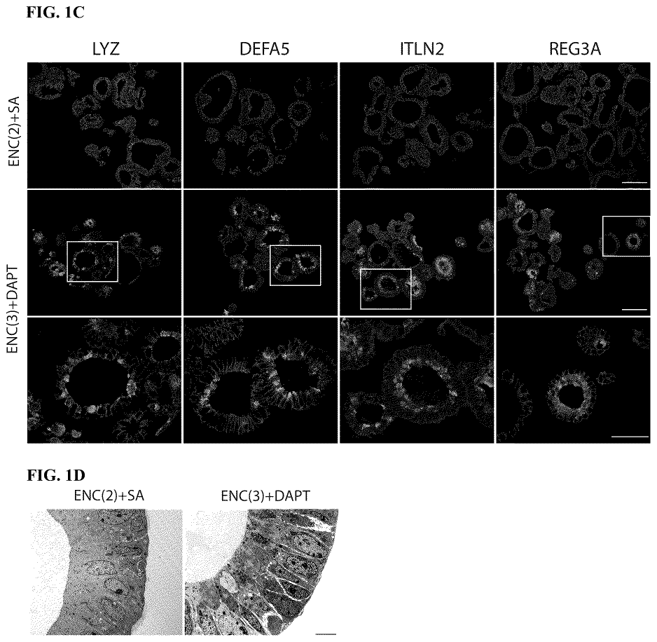

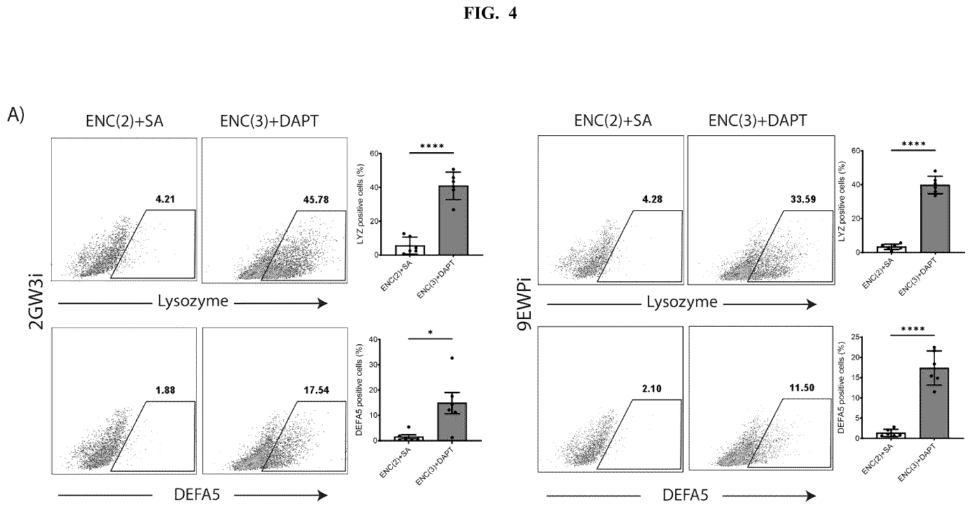

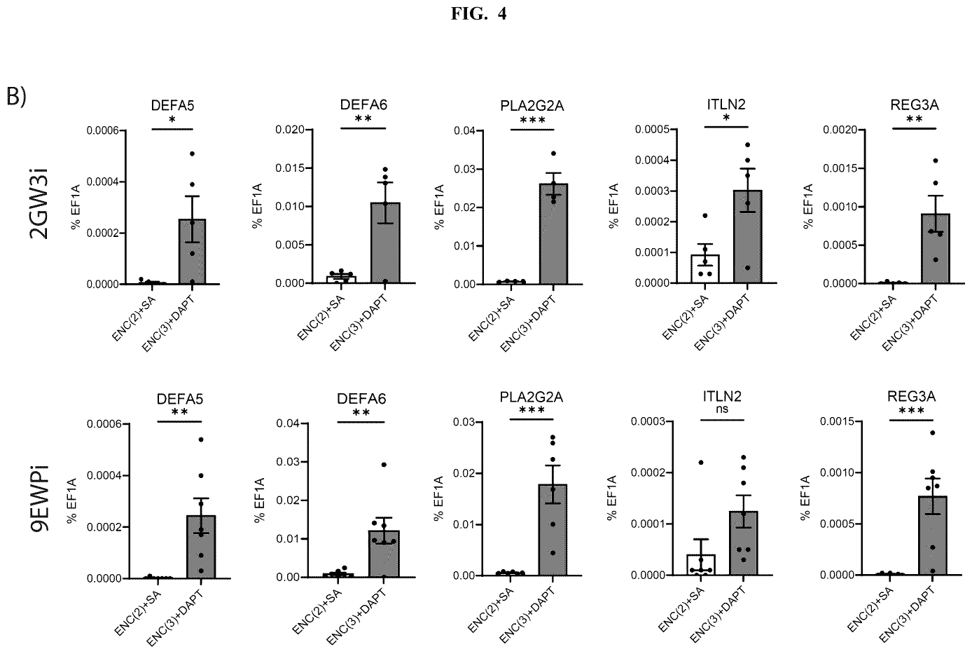

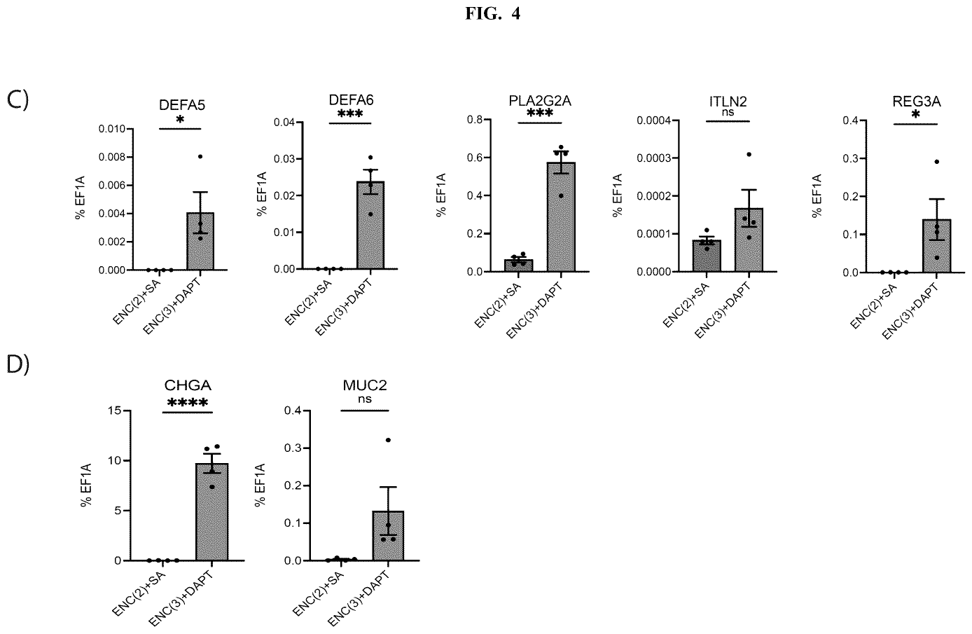

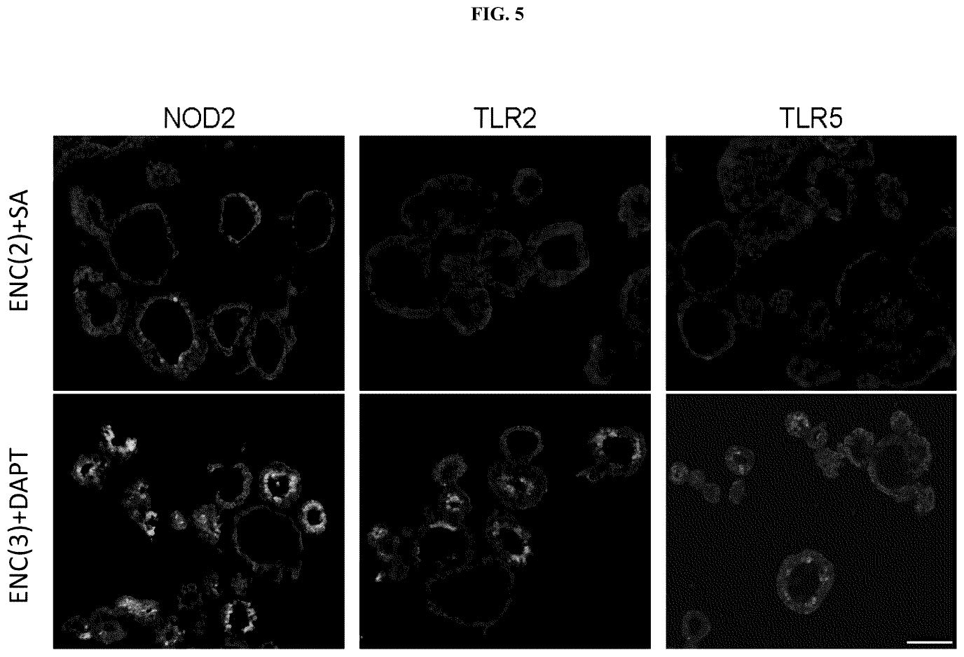

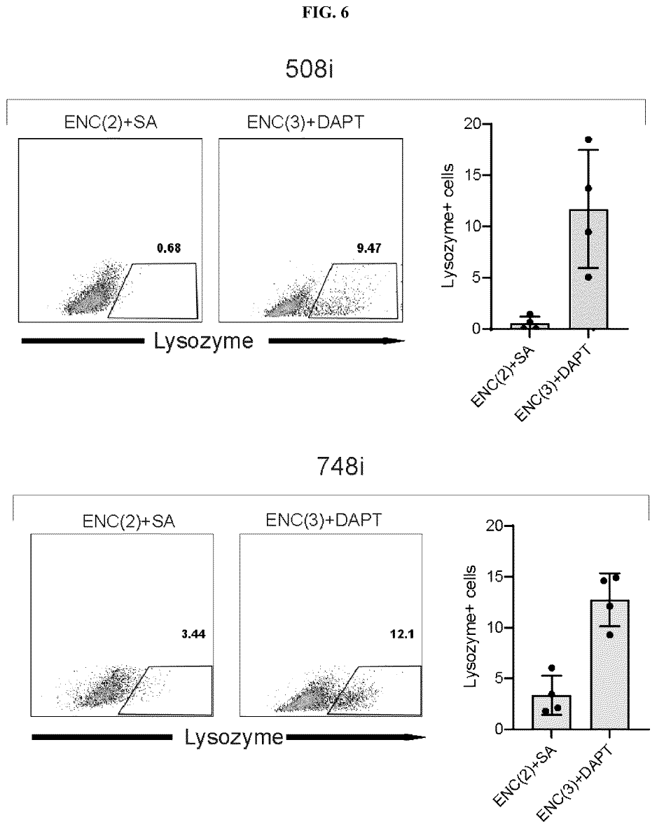

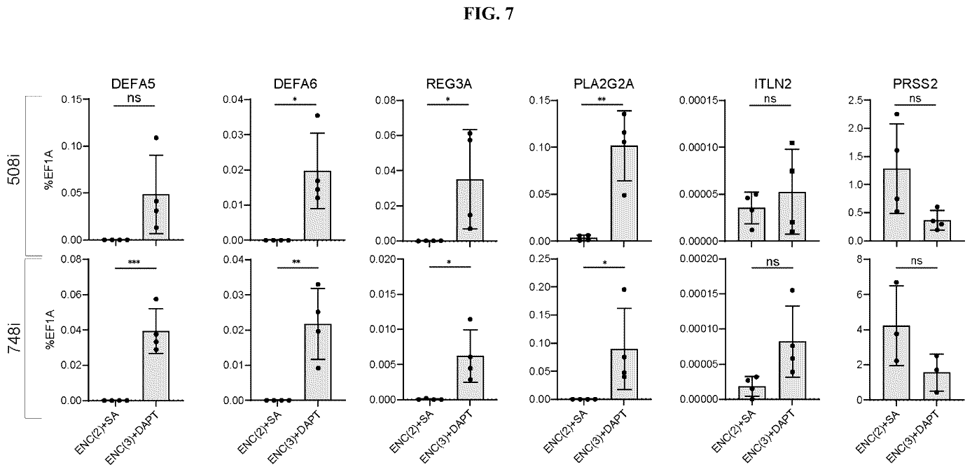

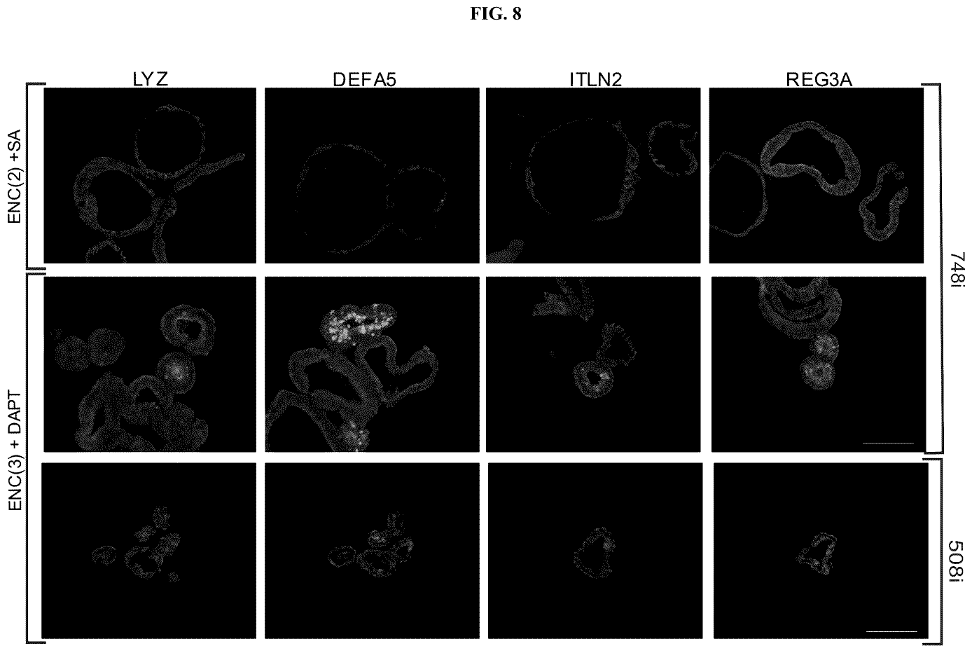

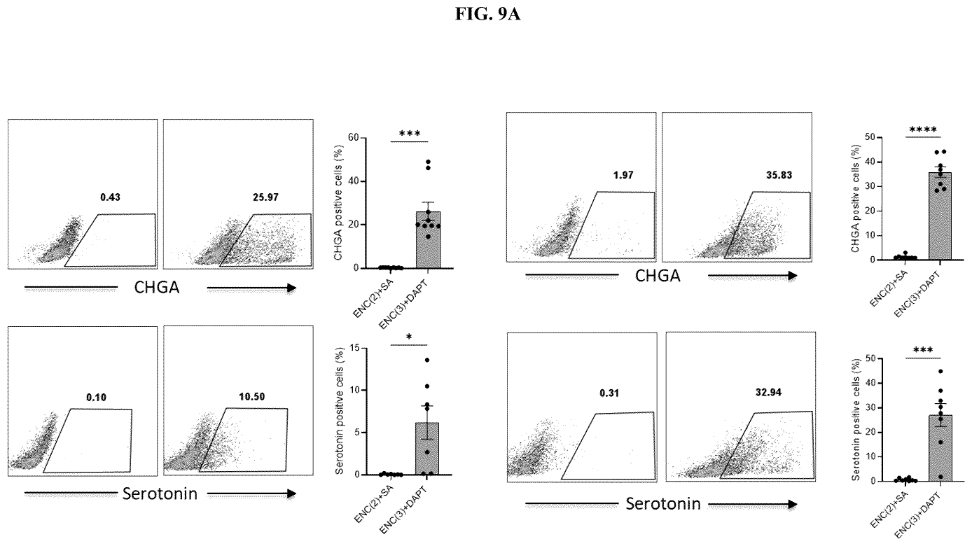

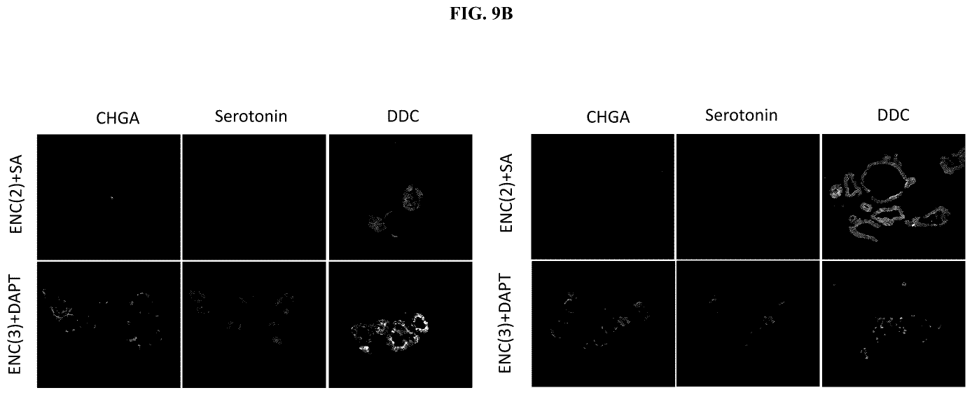

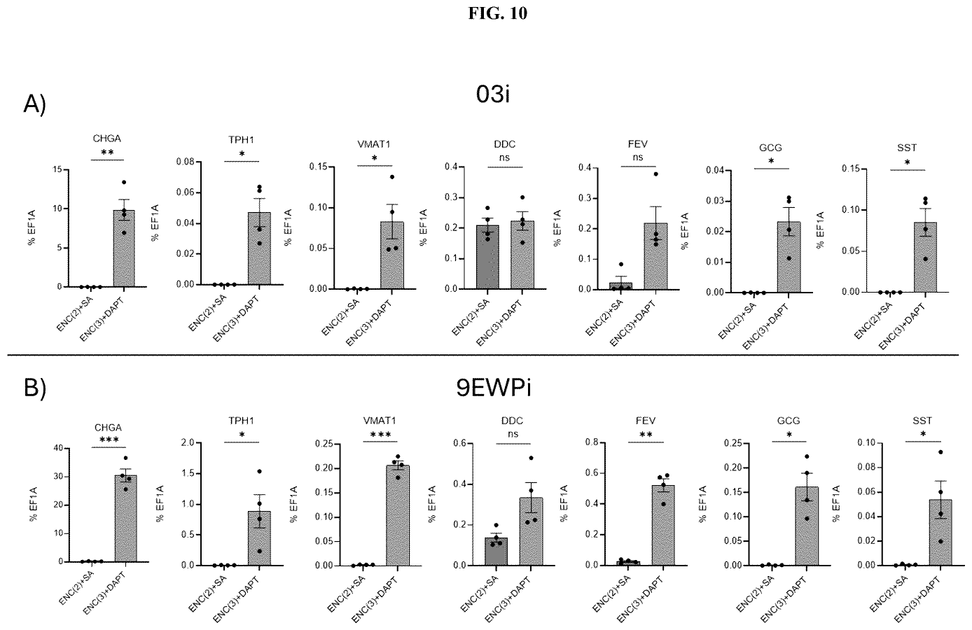

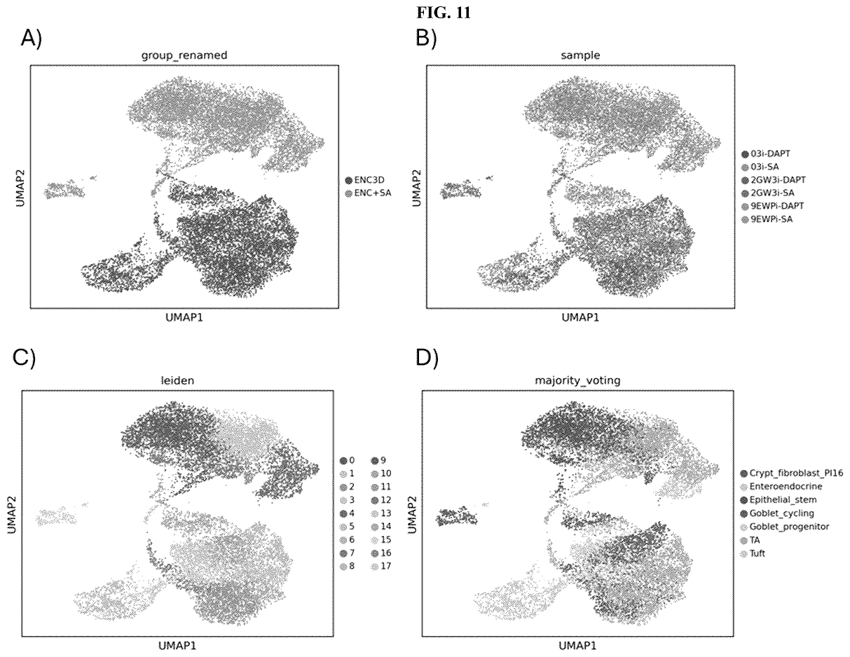

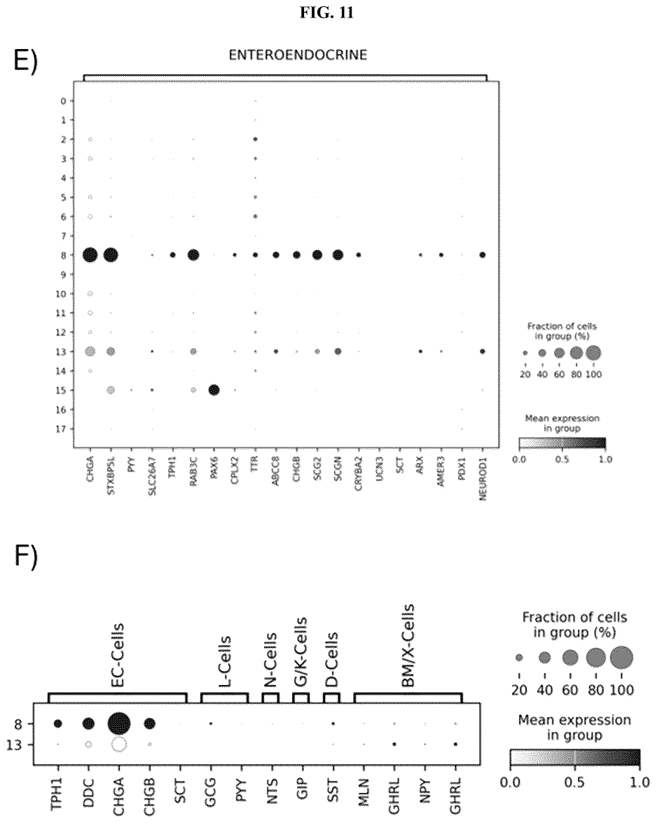

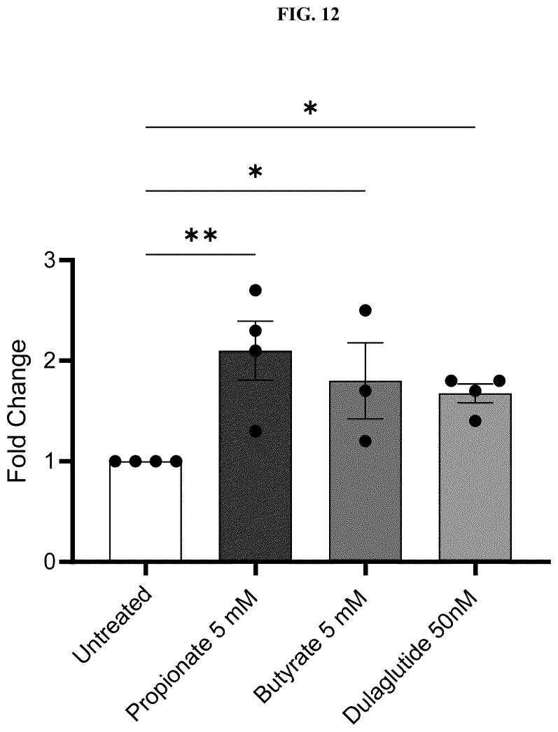

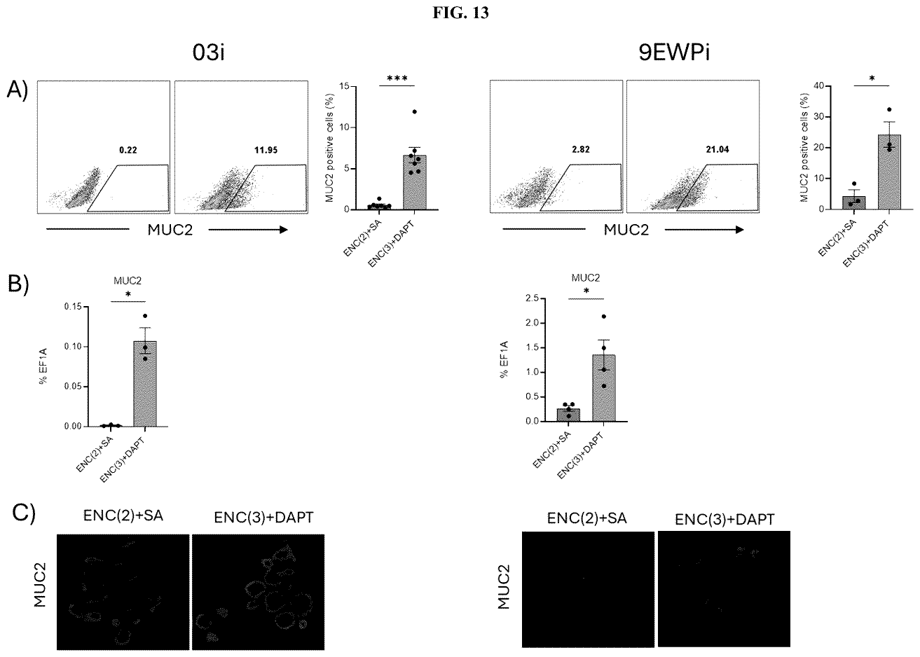

Previous attempts to enrich for Paneth cells in the organoid modeling system generally utilized the Notch inhibitor, DAPT, and the GSK-inhibitor CHIR99021 and while this resulted in increased numbers of lysozyme+ cells (~20-85%) in murine intestinal organoids, human biopsy-derived organoids were generally limited to increases in the expression of Paneth cell related genes. Two studies attempted to enrich via IL-22 addition in biopsy-derived human intestinal organoids and this resulted in the presence of ~1-15% Paneth cells. To assess both of these approaches in our iPSC-derived cells, we first modified our eHIO maintenance media and found the removal of the small molecules SB2021901/A83-01 and increased concentration of CHIR99021 (2 mM→3 mM) led to a significant increase in lysozyme+ cells from ~2.5% to ~10%, and while there was no significant increase with the addition of IL-22, there was a significant increase to ~30% lysozyme+ cells with the addition of DAPT (FIG. 1A). We then assessed for previously identified human Paneth cell related genes and found that increased CHIR99021 and DAPT led to a significant increase in the expression of DEFA5, DEFA6, PLA2G2A, REG3A, and ITLN2 mRNA (FIG. 1B) as compared to those in maintenance media. We then confirmed via immunofluorescent stainings that these factors led to numerous cells within eHIOs to possess not only granulated lysozyme, but also granulated DEFA5, REG3A, and ITLN2 (FIG. 1C) and transmission electron microscopy revealed the presence of electron dense granules (FIG. 1D) all of which suggests the presence of an enriched Paneth cell population. Given Notch inhibition can globally influence secretory differentiation, we also observed increased expression of both CHGA and MUC2 which suggests that both enteroendocrine and goblet cells are obtained also (FIG. 4C). Finally, to confirm that this protocol is not restricted to the aforementioned control iPSC line, we demonstrated that the addition of increased CHIR99021 and DAPT to eHIOs, generated from 2 other control iPSC lines. Under ENC(3)+DAPT conditions, CS2GW3i eHIOs contained 45.8% LYZ⁺ and 17.5% DEFA5⁺ cells, whereas CS9EWPi eHIOs showed 33.6% LYZ⁺ and 11.5% DEFA5⁺ cells (FIG. 4A) and there was significantly upregulated DEFA5, DEFA6, PLA2G2A, and REG3A in both lines (FIG. 4B).

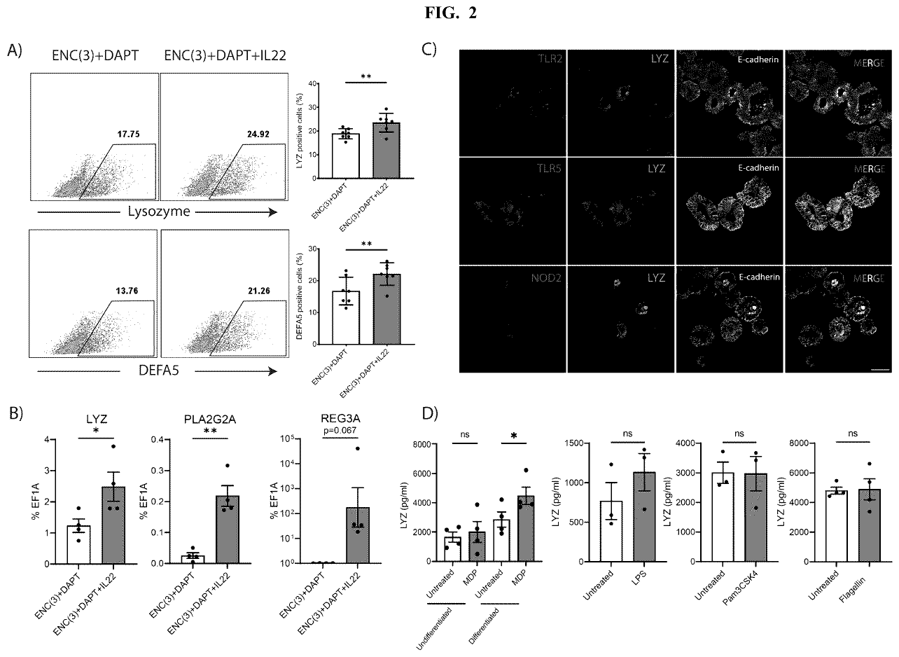

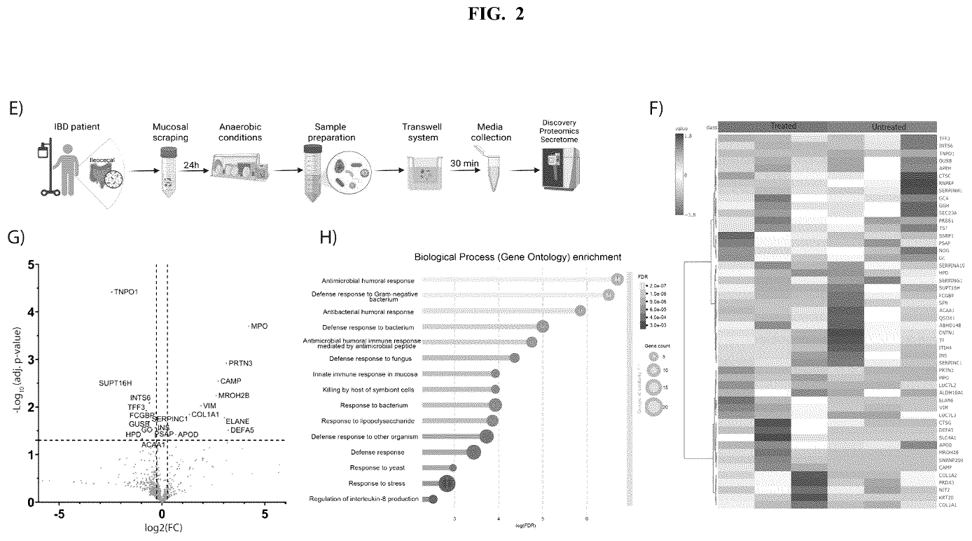

Having established the protocol to enrich the Paneth cell population in eHIOs, we then wished to confirm that these cells are biologically responsive to their milieu. Given that Paneth cells are at the interface between the mucosal immune system and microbiome, we wished to examine the responses to each. Firstly, we examined the effects of IL-22 in the organoid modality for 5 days and found there was significant increase in both the lysozyme+ and HD5+ population of cells (FIG. 2A). Furthermore, although there were no changes in the expression of DEFA5 and DEFA6, there were significant increases in the expression of LYZ and PLA2G2A (FIG. 2B), thereby demonstrating their responsivity to cytokines.

As Paneth cell responses to microbial ligands/microbes are of considerable interest, we aimed to investigate such responses. We first investigated for the presence of various bacterial sensors in enriched Paneth cell-eHIOs and confirmed the presence of TLR2, TLR5, and NOD2 (FIG. 2C). As intestinal organoids are polarized towards the lumen, we dissociated enriched organoids and seeded them onto Transwells whereby the luminal aspect could be accessed so as to examine the effects of both microbial ligands and live bacteria and also quantify the secreted AMPs via ELISA and mass spectrometry. We confirmed that Paneth cell markers remained highly enriched after seeding onto Transwells indicating these cells were retained upon seeding (FIG. 4C) and also suggests the additional secretory cell types were present also (FIG. 4D). We tested for ligands of the aforementioned receptors and found that the NOD2 ligand MDP caused a significant increase in the secretion of lysozyme (FIG. 2D) but not by the Pam3CSK4, Flagellin, or LPS which is similar to a previous study. Importantly, MDP did not induce lysozyme secretion in undifferentiated eHIOs, indicating that this response is specific to the Paneth cell-enriched state Finally, we co-cultured live bacteria, obtained from resected ileocolonic tissue (for schematic see FIG. 2E), in Transwells with Paneth cell enriched monolayers and found via mass spectrometry a significant increase in DEFA5 secretion, among others (FIG. 2F, G) which additionally underscores a Paneth cell-specific response. To identify pathways altered by bacterial exposure, we performed Gene Ontology (GO) Biological Process enrichment analysis using STRING (version 12.0) in significantly changed secreted proteins (FDR<0.05). Consistent with the analysis, “Antimicrobial humoral response” and “Defense response to bacterium” were among the most upregulated pathways (FIG. 2H).

Our goal was to enrich the Paneth cell population in eHIOs, generated from iPSCs, and we demonstrate that we have achieved this by illustrating the presence of AMPs and their responses to various stimuli. Given that iPSCs can be generated from almost all individuals, this now permits a methodology whereby Paneth cells from a range of individuals, including those with Crohn's disease among others, can be generated and thus allows for studies into how genetic variations, microbial ligand/microbes and components of the mucosal immune system influence this cell type.

We have the ability to generate human intestinal organoids, derived from induced pluripotent stem cells, from control or Crohn's disease patients and we can direct them to possess enriched populations of Paneth cells, as well as enterochromaffin and goblet cells. These cells are ultimately responsive to their milieu. Enterochromaffin cells are a subtype of enteroendocrine and they produce 90-95% of a person's serotonin. When assessed via scRNAseq, we found that we had a very strong signature for enterochromaffin cells. In addition, enterochromaffin cells are responsive to GLP1-R agonists and we found that one (dulaglutide) caused a significant increase in the secretion of serotonin from this cell type in transwell culture.

Described herein, we are the first to be able to enrich iPSC-derived human intestinal organoids for Paneth cells, as well as enterochromaffin and goblet cells. We can also dissociate them and seed onto transwells so we can assess both luminal and basal administration of stimuli.

We have generated human intestinal organoids from a control individual and significantly enhanced the Paneth population by the additional of CHIR99021 and DAPT, a gamma secretase inhibitor. We observe significant increases in the number of Paneth cells in our organoids, they have a significant upregulation of Paneth cell related genes (HD5, HD6, PLA2AG2A, REG3A) and can observe the presence of lysozyme, HD5, REG3A etc. We also show the presence of electron dense granules via immunocytochemistry.

Our invention allows iPSC-derived organoids to give rise to an enhanced population of Paneth cells. The iPSC-derived approach allows donor cells from CD patients with very specific genotypes to be identified and ultimately give rise to enhanced Paneth cell organoids. Biopsy-derived organoids require colonoscopy so specific genotypes may not be able to be obtained if such patients do not require this procedure. Our invention allow cells to be placed on transwells so both the luminal and basal aspects of Paneth cells can be exposed to various stimuli. Finally, it should be emphasized that described herein is a human model while most Paneth cell studies have been carried out in mice.

We have a highly reproducible system in which to generate these cells and routinely generate them every week. While the addition of CHIR and DAPT has frequently been used in the murine models, there is no study reporting this in human cells. The key points are age of the organoids and how long you culture them in vitro.

Epithelial-only human intestinal organoids, derived from iPSCs, can be enriched towards a Paneth cell. iPSC-derived Paneth cells have increased expression of Paneth cell related genes and presence of antimicrobial peptides.

This modeling system now allows for the study of human Paneth cells under controlled culturing conditions. It also allows for a study of how genetic variations associated with inflammatory bowel disease influences Paneth cells. It further allows for studies examining how environmental factors (cytokines, microbes, etc.) influence Paneth cell function.

Described herein is a methodology to enrich Paneth cells, as well as enterochromaffin and goblet cells, in iPSC-derived HIOs from both healthy and CD patients. Given that iPSCs can be generated from donor cells stored in well characterized biorepositories or obtained from a small blood draw from any CD patient, this modeling system now opens up a new avenue of research by allowing an examination of how environmental factors (microbes/cytokines) and/or genetic variations influence human Paneth cell function in a personalized manner.

Various embodiments of the invention are based, at least in part, on these findings.

Differentiating eHIOs into Paneth Cells and Enriching the Paneth Cells

Various embodiments provide for a method of differentiating epithelial human intestinal organoids into Paneth Cells and enriching the Paneth Cells in eHIO culture, comprising: seeding about 10,000-100,000 human epithelial intestinal cells into a MATRIGEL bubble and maintaining the human epithelial intestinal cells in eHIO medium for about 1-4 days; replacing the eHIO medium with differentiation medium and culturing for about 3-7 days; replating the human epithelial intestinal cells and continuing to culture in differentiation medium for an additional 3-7 days thereby generating a cell culture enriched with Paneth cells. In various embodiments, the method further comprises harvesting cells from the cell culture enriched with Paneth cells.

In various embodiments, the method of differentiating epithelial human intestinal organoids into Paneth Cells and enriching the Paneth Cells in eHIO culture, comprises: seeding about 25,000-75,000 human epithelial intestinal cells into a MATRIGEL bubble and maintaining the human epithelial intestinal cells in eHIO medium for about 1-3 days; replacing the eHIO medium with differentiation medium and culturing for about 4-6 days; replating the human epithelial intestinal cells and continuing to culture in differentiation medium for an additional 4-6 days thereby generating a cell culture enriched with Paneth cells. In various embodiments, the method further comprises harvesting cells from the cell culture enriched with Paneth cells.

In various embodiments, the method of differentiating epithelial human intestinal organoids into Paneth Cells and enriching the Paneth Cells in eHIO culture, comprises: seeding about 40,000-60,000 human epithelial intestinal cells into a MATRIGEL bubble and maintaining the human epithelial intestinal cells in eHIO medium for about 1.5-2.5 days; replacing the eHIO medium with differentiation medium and culturing for about 4.5-5.5 days; replating the human epithelial intestinal cells and continuing to culture in differentiation medium for an additional 4.5-5.5 days thereby generating a cell culture enriched with Paneth cells. In various embodiments, the method further comprises harvesting cells from the cell culture enriched with Paneth cells.

In various embodiments, the method comprises: seeding about 50,000 human epithelial intestinal cells into a Matrigel bubble and maintaining the human epithelial intestinal cells in eHIO medium for about 2 days; replacing the eHIO medium with differentiation medium and culturing for about 5 days; replating the human epithelial intestinal cells and continuing culture in differentiation medium for an additional 5 days thereby generating a cell culture enriched with Paneth cells; and harvesting cells from the cell culture enriched with Paneth cells.

In various embodiments, the method further comprises analyzing Paneth cell numbers and gene expression and/or protein expression using flow cytometry, qPCR, immunocytochemistry, or combinations thereof.

In various embodiments, the Paneth cells have an increased mRNA expression of one or more genes selected from the group consisting of DEFA5, DEFA6, PLA2G2A, REG3A, and ITLN2, as compared to undifferentiated eHIOs. In various embodiments, the Paneth cells have an increased mRNA expression of DEFA5, DEFA6, PLA2G2A, and REG3A, as compared to undifferentiated eHIOs. In various embodiments, the Paneth cells have an increased mRNA expression of HD5, HD6, PLA2AG2A, and REG3A, and have the presence of lysozyme, HD5, REG3A, as compared to undifferentiated eHIOs. In various embodiments, the cell culture comprising Paneth cells show a presence of electron dense granules via immunocytochemistry. In various embodiments, the Paneth cells are LYZ⁺ and DEFA5⁺.

In various embodiments, the epithelial human intestinal organoids are generated by methods as described herein.

In various embodiments, the human epithelial intestinal cells are differentiated from human induced pluripotent stem cells (iPSCs). Exemplary methods of differentiating iPSCs into human epithelial intestinal cells are described herein.

In various embodiments, the human epithelial intestinal cells are human epithelial hindgut cells differentiated from human iPSCs. Exemplary methods of differentiating iPSCs into human epithelial hindgut cells are described herein.

In various embodiments, the epithelial human intestinal organoids (eHIOs) are differentiated from human iPSCs. Exemplary methods of differentiating iPSCs into eHIOs are described herein.

In various embodiments, the eHIO medium comprises Adv DMEM/F12, L-glut, B27, EGF, Noggin, CHIR, SB202190, A8301, and optionally, Rock inhibitor, and Penicillin-Streptomycin. In various embodiments, the CHIR is at a concentration of at least 3 mM. In various embodiments, the concentration of CHIR99021 is at least 4 μM, 5 μM, 6 μM, 7 μM, 8 μM, 9 μM or 10 μM. In various embodiments, the concentration of CHIR99021 is about 3 μM. In various embodiments, the concentration of CHIR99021 is about 4 μM, about 5 μM, about 6 μM, about 7 μM, about 8 μM, about 9 μM or about 10 μM. Exemplary differentiation medium is as provided herein. In various embodiments, the eHIO medium is as further provided herein.

In various embodiments, differentiation medium comprises Adv DMEM/F12, Penicillin-Streptomycin, 200 mM L-glut, B27, EGF, Noggin, CHIR, and DAPT.

In various embodiments, the differentiation medium comprises CHIR99021 and DAPT.

In various embodiments, the concentration of CHIR99021 is at least 3 μM. In various embodiments, the concentration of CHIR99021 is at least 4 μM, 5 μM, 6 μM, 7 μM, 8 μM, 9 μM or 10 μM. In various embodiments, the concentration of CHIR99021 is about 3 μM. In various embodiments, the concentration of CHIR99021 is about 4 μM, about 5 μM, about 6 μM, about 7 μM, about 8 μM, about 9 μM or about 10 μM. Exemplary differentiation medium is as provided herein.

In various embodiments, the concentration of DAPT is at least 5 μM. In various embodiments, the concentration of DAPT is at least 10 μM. In various embodiments, the concentration of DAPT is at least 5 μM, 6 μM, 7 μM, 8 μM, 9 μM or 10 μM. In various embodiments, the concentration of DAPT is about 5 μM. In various embodiments, the concentration of DAPT is about 10 μM. In various embodiments, the concentration of DAPT is about 10-15 μM. In various embodiments, the concentration of DAPT is about 10-12 μM. In various embodiments, the concentration of DAPT is about 15-20 μM. In various embodiments, the concentration of CHIR99021 is about 5 μM, about 6 μM, about 7 μM, about 8 μM, about 9 μM, about 10 μM, about 11 μM, about 12 μM, about 13 μM, about 14 μM, about 15 μM. Exemplary differentiation medium is as further provided herein.

In various embodiments, the differentiation medium further comprise IL-22.

In various embodiments, the human epithelial intestinal cells are differentiated from human intestinal organoids (HIOs) that have been cultured for about 15-50 days before being sorted and grown as epithelial only-human intestinal organoids. In various embodiments, the human epithelial intestinal cells are differentiated from human intestinal organoids (HIOs) that have been cultured for about 25-35 days before being sorted and grown as epithelial only-human intestinal organoids. In various embodiments, the human epithelial intestinal cells are differentiated from human intestinal organoids (HIOs) that have been cultured for at least 30 days before being sorted and grown as epithelial only-human intestinal organoids. In various embodiments, the human epithelial intestinal cells are differentiated from human intestinal organoids (HIOs) that have been cultured for about 30 days before being sorted and grown as epithelial only-human intestinal organoids.

In various embodiments, the human epithelial intestinal cells are from epithelial only-human intestinal organoids (eHIOs) that have been passaged for at least 5 times. In various embodiments, the human epithelial intestinal cells are from epithelial only-human intestinal organoids (eHIOs) that have been passaged for at least 10 times. In various embodiments, the human epithelial intestinal cells are from epithelial only-human intestinal organoids (eHIOs) that have been passaged for at least 15 times. In various embodiments, the human epithelial intestinal cells are from epithelial only-human intestinal organoids (eHIOs) that have been passaged for about 5-30 times. In various embodiments, the human epithelial intestinal cells are from epithelial only-human intestinal organoids (eHIOs) that have been passaged for about 10-15 times.

In various embodiments, the epithelial human intestinal cells are obtained by:
removing HIOs from MATRIGEL and washing the HIOs;
dissociating the HIOs into dissociated cells;
separating and isolating EpCAM+ cells;
resuspending EpCAM+ cells in MATRIGEL as single cells, wherein the EpCAM+ cells self-organize and expand to form epithelial-only HIOs (eHIOs);
maintaining the eHIOs in organoid medium supplemented with SB202190 and A83-01; and
dissociating the eHIOs into single cell suspension.

In various embodiments, washing the HIOs comprises washing 3 times in Dulbecco's Phosphate-Buffered Saline.

In various embodiments, dissociating the HIOs into dissociated cells comprises incubating the HIOs in cell detachment enzyme to dissociate the cells. In various embodiments, dissociating the HIOs into dissociated cells comprises incubating the HIOs in TrypLE™ Select to dissociate the cells. Incubating can be performed to about 6-18 minutes, about 8-16 minutes, 10-14 minutes, or about 12 minutes; alternatively, incubating can be performed until the organoids were at least 95%, at least 96%, at least 97%, at least 98%, at least 99%, or completely dissociated to a single-cell suspension.

In various embodiments, separating and isolating EpCAM+ cells comprises incubating the dissociated cells with EpCAM MicroBeads. Incubating the dissociated cells can be performed for at least 10 minutes, at least 20 minutes or at least 30 minutes; or about 10-60 minutes, or 20-50 minutes, or 30-40 minutes. Incubation temperature can be at about 3° C., about 4° C., about 5° C., about 6° C., about 7° C., about 8° C., about 9° C., or about 10° C.; or at about 3-5° C., 3-10° C., 3-15° C., 3-20° C., or 3-30° C.

In various embodiments, the method generates at least 30% Paneth cells. In various embodiments, the method generates about 30% Paneth cells. In various embodiments, the method generates at least 15%, 20%, 25%, 30% or 35% Paneth cells. In various embodiments, the method generates about 15%, 20%, 25%, 30% or 35% Paneth cells.

Various embodiments of the invention provide for epithelial human intestinal organoids (eHIOs) enriched for human Paneth cells generated by one or more methods described herein.

Various embodiments of the invention provide for a cell culture enriched for human Paneth cells generated by one or more methods described herein.

Various embodiments of the invention provide for meaningful numbers of human Paneth cells generated by one or more methods described herein. In various embodiments, meaningful numbers of human Paneth cells comprise generating at least 30% Paneth cells. In various embodiments, meaningful numbers of human Paneth cells comprise generating at about 30% Paneth cells. In various embodiments, meaningful numbers of human Paneth cells comprise generating at least 15%, 20%, 25%, 30% or 35% Paneth cells. In various embodiments, meaningful numbers of human Paneth cells comprise generating at about 15%, 20%, 25%, 30% or 35% Paneth cells.

Various embodiments of the invention provide for human Paneth cells obtained by any one of the methods described herein.

Various embodiments of the invention provide for human Paneth cell organoids obtained by any one of the methods described herein.

Various embodiments of the invention provide for a human Paneth cell organoid model comprising: human Paneth cell organoids obtained by any one of the methods described herein.

In various embodiments, the Paneth cells have an increased mRNA expression of one or more genes selected from the group consisting of DEFA5, DEFA6, PLA2G2A, REG3A, and ITLN2, as compared to undifferentiated eHIOs. In various embodiments, the Paneth cells have an increased mRNA expression of DEFA5, DEFA6, PLA2G2A, and REG3A, as compared to undifferentiated eHIOs. In various embodiments, the Paneth cells have an increased mRNA expression of HD5, HD6, PLA2AG2A, and REG3A, and have the presence of lysozyme, HD5, REG3A, as compared to undifferentiated eHIOs. In various embodiments, the cell culture comprising Paneth cells show a presence of electron dense granules via immunocytochemistry. In various embodiments, the Paneth cells are LYZ⁺ and DEFA5⁺. Paneth cells are notoriously difficult for scRNAseq processing and thus, we show conclusively that Paneth cells are present through a variety of methods discussed herein.

Differentiating eHIOs into Enterochromaffin Cells, Goblet Cells or Both

Various embodiments provide for a method of differentiating epithelial human intestinal organoids into enterochromaffin cells, goblet cells or both and enriching the enterochromaffin cells, goblet cells, or both in eHIO culture, comprising:
seeding about 25,000-75,000 human epithelial intestinal cells into a MATRIGEL bubble and maintaining the human epithelial intestinal cells in eHIO medium for about 1-3 days;
replacing the eHIO medium with differentiation medium and culturing for about 4-6 days;
replating the human epithelial intestinal cells and continuing to culture in differentiation medium for an additional 4-6 days thereby generating a cell culture enriched with enterochromaffin cells, goblet cells, or both.

In various embodiments, the enterochromaffin cells are CHGA⁺ and serotonin⁺. In various embodiments, the enterochromaffin cells are CHGA⁺, TPH1⁺, VMAT1⁺, and DDC⁺. In various embodiments, the goblet cells have increased MUC2 expression as compared to undifferentiated eHIOs. In various embodiments, the enterochromaffin cells secrete serotonin. In various embodiments, the enterochromaffin cells are responsive to GLP1-R agonists. For example, the enterochromaffin cells increase serotonin secretion in response to GLP1-R agonists.

In various embodiments, the method further comprises c harvesting enterochromaffin cells, goblet cells, or both from the cell culture enriched with enterochromaffin cells, goblet cells, or both.

In various embodiments, the method comprises:
seeding about 35,000-65,000 human epithelial intestinal cells into a MATRIGEL bubble and maintaining the human epithelial intestinal cells in eHIO medium for about 1.5-2.5 days;
replacing the eHIO medium with differentiation medium and culturing for about 4.5-5.5 days;
replating the human epithelial intestinal cells and continuing to culture in differentiation medium for an additional 4.5-5.5 days thereby generating a cell culture enriched with enterochromaffin cells, goblet cells, or both.

In various embodiments, the enterochromaffin cells are CHGA⁺ and serotonin⁺. In various embodiments, the enterochromaffin cells are CHGA⁺, TPH1⁺, VMAT1⁺, and DDC⁺. In various embodiments, the goblet cells have increased MUC2 expression as compared to undifferentiated eHIOs. In various embodiments, the enterochromaffin cells secrete serotonin. In various embodiments, the enterochromaffin cells are responsive to GLP1-R agonists. For example, the enterochromaffin cells increase serotonin secretion in response to GLP1-R agonists.

In various embodiments, the method further comprises harvesting enterochromaffin cells, goblet cells, or both from the cell culture enriched with enterochromaffin cells, goblet cells, or both

In various embodiments, the method comprises:
seeding about 50,000 human epithelial intestinal cells into a Matrigel bubble and maintaining the human epithelial intestinal cells in eHIO medium for about 2 days;
replacing the eHIO medium with differentiation medium and culturing for about 5 days;
replating the human epithelial intestinal cells and continuing culture in differentiation medium for an additional 5 days thereby generating a cell culture enriched with enterochromaffin cells, goblet cells, or both.

In various embodiments, the epithelial human intestinal organoids are generated by methods as described herein.

In various embodiments, the human epithelial intestinal cells are differentiated from human induced pluripotent stem cells (iPSCs). Exemplary methods of differentiating iPSCs into human epithelial intestinal cells are described herein.

In various embodiments, the human epithelial intestinal cells are human epithelial hindgut cells differentiated from human iPSCs. Exemplary methods of differentiating iPSCs into human epithelial hindgut cells are described herein.

In various embodiments, the epithelial human intestinal organoids (eHIOs) are differentiated from human iPSCs. Exemplary methods of differentiating iPSCs into eHIOs are described herein.

In various embodiments, the eHIO medium comprises Adv DMEM/F12, L-glut, B27, EGF, Noggin, CHIR, SB202190, A8301, and optionally, Rock inhibitor, and Penicillin-Streptomycin. In various embodiments, the CHIR is at a concentration of at least 3 mM. In various embodiments, the concentration of CHIR99021 is at least 4 μM, 5 μM, 6 μM, 7 μM, 8 μM, 9 μM or 10 μM. In various embodiments, the concentration of CHIR99021 is about 3 μM. In various embodiments, the concentration of CHIR99021 is about 4 μM, about 5 μM, about 6 μM, about 7 μM, about 8 μM, about 9 μM or about 10 μM. Exemplary differentiation medium is as provided herein. In various embodiments, the eHIO medium is as further provided herein.

In various embodiments, differentiation medium comprises Adv DMEM/F12, Penicillin-Streptomycin, 200 mM L-glut, B27, EGF, Noggin, CHIR, and DAPT.

In various embodiments, the differentiation medium comprises CHIR99021 and DAPT.

In various embodiments, the concentration of CHIR99021 is at least 3 μM. In various embodiments, the concentration of CHIR99021 is at least 4 μM, 5 μM, 6 μM, 7 μM, 8 μM, 9 μM or 10 μM. In various embodiments, the concentration of CHIR99021 is about 3 μM. In various embodiments, the concentration of CHIR99021 is about 4 μM, about 5 μM, about 6 μM, about 7 μM, about 8 μM, about 9 μM or about 10 μM. Exemplary differentiation medium is as provided herein.

In various embodiments, the concentration of DAPT is at least 5 μM. In various embodiments, the concentration of DAPT is at least 10 μM. In various embodiments, the concentration of DAPT is at least 5 μM, 6 μM, 7 μM, 8 μM, 9 μM or 10 μM. In various embodiments, the concentration of DAPT is about 5 μM. In various embodiments, the concentration of DAPT is about 10 μM. In various embodiments, the concentration of DAPT is about 10-15 μM. In various embodiments, the concentration of DAPT is about 10-12 μM. In various embodiments, the concentration of DAPT is about 15-20 μM. In various embodiments, the concentration of CHIR99021 is about 5 μM, about 6 μM, about 7 μM, about 8 μM, about 9 μM, about 10 μM, about 11 μM, about 12 μM, about 13 μM, about 14 μM, about 15 μM. Exemplary differentiation medium is as further provided herein.

In various embodiments, the differentiation medium further comprise IL-22.

In various embodiments, the human epithelial intestinal cells are differentiated from human intestinal organoids (HIOs) that have been cultured for about 15-50 days before being sorted and grown as epithelial only-human intestinal organoids. In various embodiments, the human epithelial intestinal cells are differentiated from human intestinal organoids (HIOs) that have been cultured for about 25-35 days before being sorted and grown as epithelial only-human intestinal organoids. In various embodiments, the human epithelial intestinal cells are differentiated from human intestinal organoids (HIOs) that have been cultured for at least 30 days before being sorted and grown as epithelial only-human intestinal organoids. In various embodiments, the human epithelial intestinal cells are differentiated from human intestinal organoids (HIOs) that have been cultured for about 30 days before being sorted and grown as epithelial only-human intestinal organoids.

In various embodiments, the human epithelial intestinal cells are from epithelial only-human intestinal organoids (eHIOs) that have been passaged for at least 5 times. In various embodiments, the human epithelial intestinal cells are from epithelial only-human intestinal organoids (eHIOs) that have been passaged for at least 10 times. In various embodiments, the human epithelial intestinal cells are from epithelial only-human intestinal organoids (eHIOs) that have been passaged for at least 15 times. In various embodiments, the human epithelial intestinal cells are from epithelial only-human intestinal organoids (eHIOs) that have been passaged for about 5-30 times. In various embodiments, the human epithelial intestinal cells are from epithelial only-human intestinal organoids (eHIOs) that have been passaged for about 10-15 times.

In various embodiments, the epithelial human intestinal cells are obtained by:
removing HIOs from MATRIGEL and washing the HIOs;
dissociating the HIOs into dissociated cells;
separating and isolating EpCAM+ cells;
resuspending EpCAM+ cells in MATRIGEL as single cells, wherein the EpCAM+ cells self-organize and expand to form epithelial-only HIOs (eHIOs);
maintaining the eHIOs in organoid medium supplemented with SB202190 and A83-01; and
dissociating the eHIOs into single cell suspension.

In various embodiments, washing the HIOs comprises washing 3 times in Dulbecco's Phosphate-Buffered Saline.

In various embodiments, dissociating the HIOs into dissociated cells comprises incubating the HIOs in cell detachment enzyme to dissociate the cells. In various embodiments, dissociating the HIOs into dissociated cells comprises incubating the HIOs in TrypLE™ Select to dissociate the cells. Incubating can be performed to about 6-18 minutes, about 8-16 minutes, 10-14 minutes, or about 12 minutes; alternatively, incubating can be performed until the organoids were at least 95%, at least 96%, at least 97%, at least 98%, at least 99%, or completely dissociated to a single-cell suspension.

In various embodiments, separating and isolating EpCAM+ cells comprises incubating the dissociated cells with EpCAM MicroBeads. Incubating the dissociated cells can be performed for at least 10 minutes, at least 20 minutes or at least 30 minutes; or about 10-60 minutes, or 20-50 minutes, or 30-40 minutes. Incubation temperature can be at about 3° C., about 4° C., about 5° C., about 6° C., about 7° C., about 8° C., about 9° C., or about 10° C.; or at about 3-5° C., 3-10° C., 3-15° C., 3-20° C., or 3-30° C.

In various embodiments, the method further comprises harvesting enterochromaffin cells, goblet cells, or both from the cell culture enriched with enterochromaffin cells, goblet cells, or both.

Various embodiments of the invention provide for epithelial human intestinal organoids (eHIOs) enriched for human enterochromaffin cells, goblet cells, or both generated by one or more methods described herein.

Various embodiments of the invention provide for a cell culture enriched for human enterochromaffin cells, goblet cells, or both generated by one or more methods described herein.

Various embodiments of the invention provide for meaningful numbers of human enterochromaffin cells, goblet cells, or both generated by one or more methods described herein. In various embodiments, meaningful numbers of human enterochromaffin cells, goblet cells, or both comprise generating at least 30% human enterochromaffin cells, goblet cells, or both. In various embodiments, meaningful numbers of human enterochromaffin cells, goblet cells, or both comprise generating at about 30% human enterochromaffin cells, goblet cells, or both. In various embodiments, meaningful numbers of human enterochromaffin cells, goblet cells, or both comprise generating at least 15%, 20%, 25%, 30% or 35% human enterochromaffin cells, goblet cells, or both. In various embodiments, meaningful numbers of human enterochromaffin cells, goblet cells, or both comprise generating at about 15%, 20%, 25%, 30% or 35% enterochromaffin cells, goblet cells, or both.

Various embodiments of the invention provide for human enterochromaffin cells, goblet cells, or both obtained by any one of the methods described herein.

Various embodiments of the invention provide for human enterochromaffin organoids, goblet organoids, or both obtained by any one of the methods described herein.

Various embodiments of the invention provide for human enterochromaffin organoid model, goblet organoid model, or both comprising: human enterochromaffin organoids, goblet organoids, or both obtained by any one of the methods described herein.

In various embodiments, the enterochromaffin cells are CHGA⁺ and serotonin⁺. In various embodiments, the enterochromaffin cells are CHGA⁺, TPH1⁺, VMAT1⁺, and DDC⁺. In various embodiments, the goblet cells have increased MUC2 expression as compared to undifferentiated eHIOs. In various embodiments, the enterochromaffin cells secrete serotonin. In various embodiments, the enterochromaffin cells are responsive to GLP1-R agonists. For example, the enterochromaffin cells increase serotonin secretion in response to GLP1-R agonists.

In various embodiments, the enterochromaffin cells, goblet cells, or both have increased mRNA expression of CHGA and MUC2, as compared to undifferentiated eHIOs. In various embodiments, the enterochromaffin cells are CHGA⁺ and serotonin⁺. In various embodiments, the enterochromaffin cells are CHGA⁺, TPH1⁺, VMAT1⁺, and DDC⁺. In various embodiments, the goblet cells have increased MUC2 expression as compared to undifferentiated eHIOs.

### Incorporating Enriched Human Paneth Cell Organoids in a Transwell Culture and Incorporating Enriched Human Enterochromaffin Cells, Goblet Cells, or Both, in a Transwell Culture

Various embodiments provide for a method of incorporating enriched human Paneth cell organoids in a transwell culture, comprising:
dissociating epithelial human intestinal organoids (eHIOs) enriched for human Paneth cells by: scraping a MATRIGEL bubble off a plate comprising eHIOs enriched for human Paneth cells; breaking up the MATRIGEL bubble with medium and transferring the mixture into a 15 mL conical tube; centrifuging at about 1000-2000 rpm for about 1-5 minutes; aspirating the supernatant to remove MATRIGEL; adding about 1-5 mL PBS to the pellet, breaking organoids out of the MATRIGEL, and repeating 1-3 times; centrifuging at about 1000-2000 rpm for about 1-5 minutes; removing the supernatant to remove MATRIGEL; resuspending the pellet in about 0.5-1.5 mL TrypLE Select; incubating at about 34-40° C. for about 10-14 minutes; neutralizing with about 1-3 mL of about 12-14% FBS/PBS solution; centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, the method comprises:
dissociating eHIOs enriched for human Paneth cells by using a pipette (e.g., a P1000 pipette or a 5 mL serological pipette) scrape a MATRIGEL bubble off a plate comprising eHIOs enriched for human Paneth cells; triturating the MATRIGEL bubble with medium and transferring the mixture into a 15 ml conical tube; centrifuging at about 1500 rpm for about 3 minutes; aspirating the supernatant to remove MATRIGEL but not the organoids; adding about 3 mL PBS to the pellet, triturating to break organoids out of the MATRIGEL, and repeat the wash twice; centrifuging at about 1500 rpm for about 3 minutes; aspirating the supernatant, ensuring only clean Matrigel is removed, leaving the organoids intact; resuspending the pellet in about 1 mL TrypLE Select; incubating at about 37° C. for about 12 minutes; neutralizing with about 2 mL of about 10% FBS/PBS solution; centrifuging at about 1500 rpm for about 3 minutes;
resuspending cells at a density of about 4×10⁶ cells/ml and seed about 4×10⁵ cells per transwell; and
culturing the cells for about 24 hrs.

In various embodiments, the transwells are coated with MATRIGEL for about 1-3 hrs at room temperature. In various embodiments, the transwells (about 0.33 cm²) are coated with MATRIGEL for about 2 hrs at room temperature.

In various embodiments, dissociating the eHIOs enriched for Paneth cells comprises: resuspending the pellet in about 0.5-1.5 mL TrypLE Select; incubating at about 34-40° C. for about 10-14 minutes; neutralizing with about 1-3 mL of about 12-14% FBS/PBS solution; and centrifuging at about 1000-3000 rpm for about 1-5 minutes. In various embodiments, dissociating the eHIOs enriched for Paneth cells comprises: resuspending the pellet in about 1 mL TrypLE Select; incubating at about 37° C. for about 12 minutes; neutralizing with about 2 mL of about 10% FBS/PBS solution; and centrifuging at about 1500 rpm for about 3 minutes.

In various embodiments, the method further comprises treating the cells with cytokines, microbes, or microbial ligands, or combinations thereof for about 1 to 14 days. In various embodiments, the method further comprises treating the cells with cytokines, microbes, or microbial ligands, or combinations thereof for about 5-10 days. In various embodiments, the method further comprises treating the cells with cytokines, microbes, or microbial ligands, or combinations thereof for about 24-72 hrs. Alternatively, in various embodiments, the method further comprises comprising treating the cells with cytokines, microbes, or microbial ligands, or combinations thereof for about 32-64 hrs. Alternatively, in various embodiments, the method further comprises comprising treating the cells with cytokines, microbes, or microbial ligands, or combinations thereof for about 48 hrs.

In various embodiments, the method further comprises assessing the cell responses to cytokines, microbes, or microbial ligands, or combinations thereof.

In various embodiments, the method further comprises treating the cells with glucose, tryptophan, acetate, propionate, butyrate, GLP1-R agonist combinations thereof for about 1-14 days. In various embodiments, the method further comprises treating the cells with glucose, tryptophan, acetate, propionate, butyrate, GLP1-R agonist combinations thereof for about 5-10 days. In various embodiments, the method further comprises treating the cells with glucose, tryptophan, acetate, propionate, butyrate, GLP1-R agonist combinations thereof for about 24-72 hrs. Alternatively, in various embodiments, the method further comprises comprising treating the cells with glucose, tryptophan, acetate, propionate, butyrate, GLP1-R agonist, or combinations thereof for about 32-64 hrs. Alternatively, in various embodiments, the method further comprises comprising treating the cells with glucose, tryptophan, acetate, propionate, butyrate, GLP1-R agonist, or combinations thereof for about 48 hrs. In various embodiments, the GLP1-R agonist is semaglutide, liraglutide, dulaglutide or exendin-4 (exenatide).

In various embodiments, the method further comprises assessing the cell responses to the glucose, tryptophan, acetate, propionate, butyrate, GLP1-R agonist or combinations thereof.

Various embodiments provide for a method of incorporating enriched human enterochromaffin cells, goblet cells, or both, in a transwell culture, comprising:
dissociating epithelial human intestinal organoids (eHIOs) enriched for human enterochromaffin cells, goblet cells, or both, by: scraping a MATRIGEL bubble off a plate comprising eHIOs enriched for human enterochromaffin cells, goblet cells, or both; breaking up the MATRIGEL bubble with medium and transferring the mixture into a 15 mL conical tube; centrifuging at about 1000-2000 rpm for about 1-5 minutes; aspirating the supernatant to remove MATRIGEL; adding about 1-5 mL PBS to the pellet, breaking enterochromaffin cells, goblet cells, or both out of the MATRIGEL, and repeating 1-3 times; centrifuging at about 1000-2000 rpm for about 1-5 minutes; removing the supernatant to remove MATRIGEL; resuspending the pellet in about 0.5-1.5 mL TrypLE Select; incubating at about 34-40° C. for about 10-14 minutes; neutralizing with about 1-3 mL of about 12-14% FBS/PBS solution; centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

In various embodiments, the method comprises:
dissociating eHIOs enriched for human enterochromaffin cells, goblet cells, or both, by using a pipette (e.g., P1000 pipette or a 5 mL serological pipette) scrape a MATRIGEL bubble off a plate comprising eHIOs enriched for human enterochromaffin cells, goblet cells, or both; triturating the MATRIGEL bubble with medium and transferring the mixture into a 15 ml conical tube; centrifuging at about 1500 rpm for about 3 minutes; aspirating the supernatant to remove MATRIGEL but not the enterochromaffin cells, goblet cells, or both; adding about 3 mL PBS to the pellet, triturating to break enterochromaffin cells, goblet cells, or both out of the MATRIGEL, and repeat the wash twice; centrifuging at about 1500 rpm for about 3 minutes; aspirating the supernatant, ensuring only clean Matrigel is removed, leaving the enterochromaffin cells, goblet cells, or both intact; resuspending the pellet in about 1 mL TrypLE Select; incubating at about 37° C. for about 12 minutes; neutralizing with about 2 mL of about 10% FBS/PBS solution; centrifuging at about 1500 rpm for about 3 minutes;
resuspending cells at a density of about 4×10⁶ cells/ml and seed about 4×10⁵ cells per transwell; and
culturing the cells for about 24 hrs.

In various embodiments, the transwells are coated with MATRIGEL for about 1-3 hrs at room temperature. In various embodiments, the transwells (about 0.33 cm²) are coated with MATRIGEL for about 2 hrs at room temperature.

In various embodiments, dissociating the eHIOs enriched for human enterochromaffin cells, goblet cells, or both, comprises: resuspending the pellet in about 0.5-1.5 mL TrypLE Select; incubating at about 34-40° C. for about 10-14 minutes; neutralizing with about 1-3 mL of about 12-14% FBS/PBS solution; and centrifuging at about 1000-3000 rpm for about 1-5 minutes. In various embodiments, dissociating the eHIOs enriched for human enterochromaffin cells, goblet cells, or both, comprises: resuspending the pellet in about 1 mL TrypLE Select; incubating at about 37° C. for about 12 minutes; neutralizing with about 2 mL of about 10% FBS/PBS solution; and centrifuging at about 1500 rpm for about 3 minutes.

In various embodiments, the method further comprises treating the cells with cytokines, microbes, or microbial ligands, or combinations thereof for about 1-14 days. In various embodiments, the method further comprises treating the cells with cytokines, microbes, or microbial ligands, or combinations thereof for about 5-10 days. In various embodiments, the method further comprises treating the cells with cytokines, microbes, or microbial ligands, or combinations thereof for about 24-72 hrs. Alternatively, in various embodiments, the method further comprises comprising treating the cells with cytokines, microbes, or microbial ligands, or combinations thereof for about 32-64 hrs. Alternatively, in various embodiments, the method further comprises comprising treating the cells with cytokines, microbes, or microbial ligands, or combinations thereof for about 48 hrs.

In various embodiments, the method further comprises assessing the cell responses to cytokines, microbes, or microbial ligands, or combinations thereof.

In various embodiments, the method further comprises treating the cells with glucose, tryptophan, acetate, propionate, butyrate, GLP1-R agonist combinations thereof for about 1-14 days. In various embodiments, the method further comprises treating the cells with glucose, tryptophan, acetate, propionate, butyrate, GLP1-R agonist combinations thereof for about 5-10 days. In various embodiments, the method further comprises treating the cells with glucose, tryptophan, acetate, propionate, butyrate, GLP1-R agonist combinations thereof for about 24-72 hrs. Alternatively, in various embodiments, the method further comprises comprising treating the cells with glucose, tryptophan, acetate, propionate, butyrate, GLP1-R agonist, or combinations thereof for about 32-64 hrs. Alternatively, in various embodiments, the method further comprises comprising treating the cells with glucose, tryptophan, acetate, propionate, butyrate, GLP1-R agonist, or combinations thereof for about 48 hrs. In various embodiments, the GLP1-R agonist is semaglutide, liraglutide, dulaglutide or exendin-4 (exenatide).

In various embodiments, the method further comprises assessing the cell responses to glucose, tryptophan, acetate, propionate, butyrate, GLP1-R agonist combinations thereof.

In various embodiments, the method is for incorporating enriched human enterochromaffin cells into a transwell culture. In various embodiments, the method is for incorporating enriched human goblet cells into a transwell culture.

Various embodiments of the present invention provide for a transwell culture obtained by any one of the methods described herein.

Various embodiments of the present invention provide for a transwell culture, comprising: the human Paneth cell organoid model obtained by any one of the methods described herein.

Various embodiments of the present invention provide for a transwell culture, comprising: the human enterochromaffin cells, goblet cells, or both obtained by any one of the methods described herein. In various embodiments the transwell culture comprises human enterochromaffin cells obtained by any one of the methods described herein. In various embodiments the transwell culture comprises human goblet cells obtained by any one of the methods described herein.

Various embodiments of the present invention provide for a transwell culture, comprising: the enterochromaffin cell model, goblet cell model, or both obtained by any one of the methods described herein.

Various embodiments of the present invention provide for a transwell culture, comprising: human Paneth cells, human enterochromaffin cells, and goblet cells.

Various embodiments of the present invention provide for a transwell culture, comprising: organoids comprising human Paneth cell cells, human enterochromaffin cells, and goblet cells.

Differentiating Induced Pluripotent Stem Cells (iPSCs) into Hindgut Cells

Various embodiments provide for method of differentiating induced pluripotent stem cells (iPSCs) into hindgut cells, comprising: adding RPMI medium supplemented with Wnt3a (about 15-30 ng/mL) and Activin A (about 75-125 ng/ml) to iPSCs in a multi-well plate at about 50-80% confluency and culturing for about 0.5-2 days; aspirating the RPMI medium supplemented with Wnt3a and Activin A, and adding RPMI medium supplemented with about 0.1-0.3% FBS and Activin A (about 75-125 ng/mL) and culturing for about 0.5-2 days; aspirating the RPMI supplemented with FBS and Activin A, and adding RPMI medium supplemented with about 0.1-0.3% FBS and Activin A (about 75-175 ng/ml) and culturing for about 0.5-2 day resulting in a cell culture comprising endoderm cells; and aspirating the RPMI supplemented with FBS and Activin A, and adding advanced DMEM/F12 medium supplemented with about 1-3% FBS, FGF4 (about 300-700 ng/ml), and CHIR (about 1-5 μM) to the cell culture comprising endoderm cells and culturing for about 2-6 days, replacing with fresh medium every 0.5-3 days, resulting in a cell monolayer comprising hindgut cells.

In various embodiments, the method comprises: adding RPMI medium supplemented with Wnt3a (about 20-30 ng/ml) and Activin A (about 80-120 ng/ml) to iPSCs in a multi-well plate at about 65-75% confluency and culturing for about 0.75-2 days; aspirating the RPMI medium supplemented with Wnt3a and Activin A, and adding RPMI medium supplemented with about 0.15-0.25% FBS and Activin A (about 80-120 ng/ml) and culturing for about 0.75-2 days; aspirating the RPMI supplemented with FBS and Activin A, and adding RPMI medium supplemented with about 0.15-0.25% FBS and Activin A (about 80-120 ng/ml) and culturing for about 0.75-2 day resulting in a cell culture comprising endoderm cells; and aspirating the RPMI supplemented with FBS and Activin A, and adding advanced DMEM/F12 medium supplemented with about 1.5-2.5% FBS, FGF4 (about 400-600 ng/ml), and CHIR (about 2-4 μM) to the cell culture comprising endoderm cells and culturing for about 3-5 days, replacing with fresh medium every 0.75-2 days, resulting in a cell monolayer comprising hindgut cells.

In various embodiments, the method comprises: adding RPMI medium supplemented with Wnt3a (about 25 ng/ml) and Activin A (about 100 ng/ml) to iPSCs in a 24-well plate at about 60-70% confluency and culturing for about 1 day; aspirating the RPMI medium supplemented with Wnt3a and Activin A, and adding RPMI medium supplemented with about 0.2% FBS and Activin A (about 100 ng/ml) and culturing for about 1 day; aspirating the RPMI supplemented with FBS and Activin A, and adding RPMI medium supplemented with about 0.2% FBS and Activin A (about 100 ng/mL) and culturing for about 1 day resulting in a cell culture comprising endoderm cells; aspirating the RPMI supplemented with FBS and Activin A, and adding advanced DMEM/F12 medium supplemented with about 2% FBS, FGF4 (about 500 ng/ml), and CHIR (about 3 μM) to the cell culture comprising endoderm cells and culturing for about 4 days, replacing with fresh medium about daily, resulting in a cell monolayer comprising hindgut cells.

Various embodiments of the invention provide for seeding and culturing human iPSCs for differentiation, comprising:
(a) preparing the multi-well plate, comprising: coating the plate with MATRIGEL by diluting about 0.5-1.5 mg in about 4-8 mL of base medium, incubating at room temperature (RT) for about 0.5-2 hours or store overnight at about 2-6° C.;
(b) detaching iPSCs, comprising: aspirating mTeSR™1 medium from the iPSC plate, adding about 0.5-1.5 mL ReLeSR™ to the well, allow it to sit for about 15-60 seconds, and then aspirate, incubating at about RT for about 2-7 minutes until the cells appear brighter under the microscope;
(c) collecting iPSCs, comprising: washing the cells with about 0.5-2 mL mTeSR™ and transferring the suspension to a 5-30 mL tube, bringing the total volume to about 10-14 mL, collecting the cell suspension and transfer 0.25-1.0 mL into each well of the coated multi-well plate; and
(d) seeding and culturing, comprising: shaking the plate to distribute the cells uniformly, maintaining the iPSCs in standard culture conditions to prepare them for differentiation into organoids.

In various embodiments, the method comprises:
(a) preparing the multi-well plate, comprising: coating the plate with MATRIGEL by diluting about 0.75-1.25 mg in about 5-6 mL of base medium, incubating at room temperature (RT) for about 0.75-1.5 hours or store overnight at about 3-5° C.;
(b) detaching iPSCs, comprising: aspirating mTeSR™1 medium from the iPSC plate, adding about 0.75-1.25 mL ReLeSR™ to the well, allow it to sit for about 20-50 seconds, and then aspirate, incubating at about RT for about 3-6 minutes until the cells appear brighter under the microscope;
(c) collecting iPSCs, comprising: washing the cells with about 0.75-1.5 mL mTeSR™ and transferring the suspension to a 10-20 mL tube, bringing the total volume to about 11-13 mL, collecting the cell suspension and transfer 0.25-0.75 mL into each well of the coated multi-well plate; and
(d) seeding and culturing, comprising: shaking the plate to distribute the cells uniformly, maintaining the iPSCs in standard culture conditions to prepare them for differentiation into organoids.

In various embodiments, the method comprises:
(a) preparing the multi-well plate, comprising: coating the plate with Matrigel® by diluting about 1 mg in about 6 mL of base medium, incubating at about room temperature (RT) for about 1 hour or store overnight at about 4° C.;
(b) detaching iPSCs, comprising: aspirating mTeSR™1 medium from the iPSC plate, adding about 1 mL ReLeSR™ to the well, allow it to sit for about 30 seconds, and then aspirate, incubate at about RT for about 5 minutes until the cells appear brighter under the microscope;
(c) collect iPSCs, comprising: washing the cells with about 1 mL mTeSR™ using a pipette (e.g., P1000 pipette) and transfer the suspension to a 15 mL tube, bringing the total volume to about 12 mL, using a serological pipette, collect the cell suspension and transfer about 0.5 mL into each well of the coated multi-well plate; and
(d) seeding and culturing, comprising: shaking the plate to distribute the cells uniformly, maintaining the iPSCs in standard culture conditions to prepare them for differentiation into organoids.

As such, in various embodiments, the iPSCs in the multi-well plate at about 50-80% confluency or about 60-70% confluency is prepared by this aforementioned method.

In various embodiments, the iPSCs in the 24-well plate at about 60-70% confluency is prepared by a method comprising:
(a) preparing the 24-well plate, comprising: coating the plate with Matrigel® by diluting about 1 mg in about 6 mL of base medium, incubating at about room temperature (RT) for about 1 hour or store overnight at about 4° C.;
(b) detaching iPSCs, comprising: aspirating mTeSR™1 medium from the iPSC plate, adding about 1 mL ReLeSR™ to the well, allow it to sit for about 30 seconds, and then aspirate, incubate at about RT for about 5 minutes until the cells appear brighter under the microscope;
(c) collect iPSCs, comprising: washing the cells with about 1 mL mTeSR™ using a pipette (e.g., P1000 pipette) and transfer the suspension to a 15 mL tube, bringing the total volume to about 12 mL, using a serological pipette, collect the cell suspension and transfer about 0.5 mL into each well of the coated 24-well plate; and
(d) seeding and culturing, comprising: shaking the plate to distribute the cells uniformly, maintaining the iPSCs in standard culture conditions to prepare them for differentiation into organoids.

As such, in various embodiments, the iPSCs in the 24-well plate at about 60-70% confluency is prepared by this aforementioned method.

### Generating Hindgut Organoids

Various embodiments of the invention provide for a method of generating hindgut organoids, comprising: without removing the medium, scraping a cell monolayer comprising hindgut cells off a plate resulting in detached cells; transferring the detached cells into a tube; breaking up the detached cells into clumps but not creating a single-cell suspension; allowing the structures to settle on ice for about 10-25 minutes to form a loose pellet. removing the medium and avoid disturbing the pellet; adding MATRIGEL to the pellet, mixing and avoiding bubbles; pipetting about 25-75 μL MATRIGEL drops into the center of each well of a multi-well plate; allowing the drops solidify for about 3-15 minutes; adding about 250-750 μL organoid medium to each well; feeding about every 2-3 days and replating organoids about every 3-10 days for about 25-40 days to allow the growth of the organoids.

In various embodiments, the method comprises: without removing the medium, scraping a cell monolayer comprising hindgut cells off a plate resulting in detached cells; transferring the detached cells into a tube; breaking up the detached cells into clumps but not creating a single-cell suspension; allowing the structures to settle on ice for about 15-20 minutes to form a loose pellet. removing the medium and avoid disturbing the pellet; adding MATRIGEL to the pellet, mixing and avoiding bubbles; pipetting about 30-70 μL MATRIGEL drops into the center of each well of a multi-well plate; allowing the drops solidify for about 4-12 minutes; adding about 400-600 μL organoid medium to each well; feeding about every 2-3 days and replating organoids about every 4-9 days for about 30-35 days to allow the growth of the organoids.

In various embodiments, the method comprises: without removing the medium, scraping a cell monolayer comprising hindgut cells off a plate using a P1000 or 5 mL pipette resulting in detached cells; transferring the detached cells into a 15 mL conical tube; triturating the detached cells with a P1000 to break up clumps but not creating a single-cell suspension; allowing the structures to settle on ice for about 15-20 minutes to form a loose pellet. aspirating the medium without disturbing the pellet; adding MATRIGEL to the pellet, mixing and, avoiding bubbles; pipetting about 50 μL MATRIGEL drops into the center of each well of a multi-well plate using a P200; allowing the drops solidify for about 5-10 minutes; adding about 500 μL organoid medium to each well; feeding about every other day and replating organoids about every 5-7 days; and growing the organoids for about 30 days.

### Preparing and Sorting Epithelial Intestinal Cells

Various embodiments provide for a method of preparing and sorting epithelial intestinal cells comprising:
(a) harvesting intestinal organoids, comprising: scraping a MATRIGEL bubble off a plate, breaking up the MATRIGEL bubble with medium and transferring the mixture into a conical tube, centrifuging at about 1000-2000 rpm for 1-5 minutes, and aspirating the supernatant to remove MATRIGEL;
(b) washing intestinal organoids, comprising: adding about 1-5 mL PBS to the pellet, breaking up intestinal organoids out of the Matrigel, and repeat the wash 1-3 times, centrifuging at about 1000-2000 rpm for about 1-5 minutes, and aspirating the supernatant to remove MATRIGEL and leaving the intestinal organoids intact;
(c) dissociating intestinal organoids, comprising: resuspending the pellet in about 0.5-1.5 mL TrypLE Select, incubating at about 34-40° C. for about 10-14 minutes, neutralizing with about 1-3 mL of about 8-12% FBS/PBS solution, and centrifuging at about 1000-2000 rpm for about 1-5 minutes;
(d) filtering and preparing for sorting, comprising: removing the supernatant and resuspending the pellet in about 1-3 mL of about 8-12% FBS/PBS solution, filtering the suspension through an about 20-40 μm filter into a new tube, centrifuging at about 1000-2000 rpm for about 1-5 minutes, aspirating the supernatant, adding about 25-75 μL EpCAM MicroBeads, about 25-75 μL Blocking Reagent, and about 250-750 μL about 10% FBS/PBS solution, mixing and incubating on ice for about 15-45 minutes, centrifuging at about 1000-2000 rpm for about 1-5 minutes, removing the supernatant, and resuspending the pellet in about 0.5-2.0 mL of about 8-12% FBS/PBS solution.

In various embodiments, the method comprises:
(a) harvesting intestinal organoids, comprising: scraping a MATRIGEL bubble off a plate, breaking up the MATRIGEL bubble with medium and transferring the mixture into a 10-30 mL conical tube, centrifuging at about 800-1800 rpm for 2-4 minutes, and aspirating the supernatant to remove MATRIGEL;
(b) washing intestinal organoids, comprising: adding about 2-4 mL PBS to the pellet, breaking up intestinal organoids out of the Matrigel, and repeat the wash 1-3 times, centrifuging at about 800-1800 rpm for about 2-4 minutes, and aspirating the supernatant to remove MATRIGEL and leaving the intestinal organoids intact;
(c) dissociating intestinal organoids, comprising: resuspending the pellet in about 0.75-1.25 mL TrypLE Select, incubating at about 35-39° C. for about 11-13 minutes, neutralizing with about 1-3 mL of about 9-11% FBS/PBS solution, and centrifuging at about 800-1800 rpm for about 2-4 minutes;
(d) filtering and preparing for sorting, comprising: removing the supernatant and resuspending the pellet in about 1-3 mL of about 9-11% FBS/PBS solution, filtering the suspension through an about 15-35 μm filter into a new tube, centrifuging at about 800-1800 rpm for about 2-4 minutes, aspirating the supernatant, adding about 30-70 μL EpCAM MicroBeads, about 30-70 μL Blocking Reagent, and about 300-700 μL about 10% FBS/PBS solution, mixing and incubating on ice for about 20-40 minutes, centrifuging at about 800-1800 rpm for about 2-4 minutes, removing the supernatant, and resuspending the pellet in about 0.5-2.0 mL of about 9-11% FBS/PBS solution.

In various embodiments, the method comprises:
(a) harvesting intestinal organoids, comprising: using a pipette (e.g., P1000 pipette or a 5 mL serological pipette) to scrape the MATRIGEL bubble off the plate, triturating the MATRIGEL bubble with medium and transferring the mixture into a 15 ml conical tube, centrifuging at about 1500 rpm for 3 minutes, and aspirating the supernatant to remove MATRIGEL, avoiding the intestinal organoids;
(b) washing intestinal organoids, comprising: adding about 3 mL PBS to the pellet, triturate vigorously to break intestinal organoids out of the Matrigel, and repeat the wash twice, centrifuging at about 1500 rpm for about 3 minutes, and aspirating the supernatant to remove MATRIGEL and leaving the intestinal organoids intact;
(c) dissociating intestinal organoids, comprising: resuspending the pellet in about 1 mL TrypLE Select, incubating at about 37° C. for about 12 minutes, neutralizing with about 2 mL of about 10% FBS/PBS solution, and centrifuging at about 1500 rpm for about 3 minutes;
(d) filtering and preparing for sorting, comprising: removing the supernatant and resuspend the pellet in about 2 mL of about 10% FBS/PBS solution, filtering the suspension through a about 30 μm filter into a new 15 mL conical tube, centrifuging at about 1500 rpm for about 3 minutes, aspirating the supernatant, adding about 50 μL EpCAM MicroBeads, about 50 μL Blocking Reagent, and about 500 μL about 10% FBS/PBS solution, mixing and incubating on ice for about 30 minutes, centrifuging at about 1500 rpm for about 3 minutes, aspirating the supernatant, and resuspending the pellet in about 1 mL of about 10% FBS/PBS solution.

In various embodiments, the method further comprises: sorting cells to obtain sorted cells comprising epithelial intestinal cells; spinning down the sorted cells comprising epithelial intestinal cells at about 1000-2000 rpm for about 1-5 minutes; seeding about 25,000-75,000 epithelial intestinal cells per well in a multi-well plate; and adding about 250-750 μL of eHIO medium.

Alternatively, in various embodiments, the method further comprises: sorting cells to obtain sorted cells comprising epithelial intestinal cells; spinning down the sorted cells comprising epithelial intestinal cells at about 800-1800 rpm for about 2-4 minutes; seeding about 30,000-70,000 epithelial intestinal cells per well in a multi-well plate; and adding about 300-700 μL of eHIO medium.

Alternatively, in various embodiments, the method further comprises: sorting cells using the autoMACS Pro system to obtain sorted cells comprising epithelial intestinal cells; spinning down the sorted cells comprising epithelial intestinal cells at about 1500 rpm for about 3 minutes; seeding about 50,000 epithelial intestinal cells per well in a 24 well plate; and adding about 500 μL of eHIO medium.

In various embodiments, harvesting epithelial intestinal organoids occur at day 20-40. In various embodiments, harvesting epithelial intestinal organoids occur at day 25-35. In various embodiments, harvesting epithelial intestinal organoids occur at day 30.

Exemplary media used in accordance with various embodiments of the invention include but are not limited to the following.

Organoid Medium

| | | | | | | | | |
|---|---|---|---|---|---|---|---|---|
| Adv DMEM/F12 | 500 | mL | 500 | mL | 400-600 | mL | 450-550 | mL |
| Penicillin-Streptomycin (100x) | 5 | mL | 2.5-7.5 | mL | 2.5-7.5 | mL | 4-6 | mL |
| 200 mM L-glut (100x) | 5 | mL | 2.5-7.5 | mL | 2.5-7.5 | mL | 4-6 | ML |
| B27 (50x) | 10 | mL | 5-15 | mL | 5-15 | mL | 8-12 | mL |
| EGF (1000x) (R&D, 236-RG | 0.5 | mL | 0.25-0.75 | mL | 0.25-0.75 | mL | 0.4-0.6 | mL |
| Noggin (1000x) (R&D, 6057-NG) | 0.5 | mL | 0.25-0.75 | mL | 0.25-0.75 | mL | 0.4-0.6 | mL |
| 2 mM CHIR (1000x) | 0.5 | mL | 0.25-0.75 | mL | 0.25-0.75 | mL | 0.4-0.6 | mL |

Organoid Medium

| Adv DMEM/F12 | — | | | | | | | |
|---|---|---|---|---|---|---|---|---|
| B27 | 1x | | 0.5x-3x | | 0.5x-2x | | 0.5x-2x | |
| CHIR | 2 | μM | 1-4 | μM | 1-3 | μM | 1.5-2.5 | μM |
| Noggin | 100 | ng/mL | 25-175 | ng/mL | 50-150 | ng/mL | 75-125 | ng/mL |
| EGF | 100 | ng/mL | 25-175 | ng/mL | 50-150 | ng/mL | 75-125 | ng/mL |
| L-glut | 1% | v/v | 0.5-4% | v/v | 0.75-3% | v/v | 0.75-2% | v/v |
| Penicillin-Streptomycin | 1% | v/v | 0.5-4% | v/v | 0.75-3% | v/v | 0.75-2% | v/v |

| eHIO Medium | | | | | | | | |
|---|---|---|---|---|---|---|---|---|
| Adv DMEM/F12 | 500 | mL | 500 | mL | 400-600 | mL | 450-550 | mL |
| Penicillin-Streptomycin (100x) | 5 | mL | 2.5-7.5 | mL | 2-8 | mL | 3-7 | mL |
| 200 mM L-glut (100x) | 5 | mL | 5-15 | mL | 2-8 | mL | 3-7 | mL |
| B27 (50x) | 10 | mL | 5-15 | mL | 5-15 | mL | 8-12 | mL |
| EGF (1000x) | 0.5 | mL | 0.25-0.75 | mL | 0.25-0.75 | mL | 0.3-0.6 | mL |
| Noggin (1000x) | 0.5 | mL | 0.25-0.75 | mL | 0.25-0.75 | mL | 0.3-0.6 | mL |
| 2 mM CHIR (1000x)* | 0.5 | mL | 0.25-0.75 | mL | 0.25-0.75 | mL | 0.3-0.6 | mL |
| SB202190 (1000x)* | 0.5 | mL | 0.25-0.75 | mL | 0.25-0.75 | mL | 0.3-0.6 | mL |
| A8301 (2000x)* | 0.25 | mL | 0.125-0.375 | mL | 0.125-0.375 | mL | 0.2-0.3 | mL |
| Rock inhibitor (1000x)** | 0.5 | mL | 0.25-0.75 | mL | 0.25-0.75 | mL | 0.3-0.6 | mL |

eHIO Medium

| Adv DMEM/F12 | — | | | | | | | |
|---|---|---|---|---|---|---|---|---|
| B27 | 1x | | 0.5x-3x | | 0.5x-2x | | 0.5x-2x | |
| CHIR | 2 | μM | 1-4 | μM | 1-3 | μM | 1.5-2.5 | μM |
| Noggin | 100 | ng/mL | 25-175 | ng/mL | 50-150 | ng/mL | 75-125 | ng/mL |
| EGF | 100 | ng/mL | 25-175 | ng/mL | 50-150 | ng/mL | 75-125 | ng/mL |
| L-glut | 1% | v/v | 0.5-4% | v/v | 0.75-3% | v/v | 0.75-2% | v/v |
| Penicillin-Streptomycin | 1% | v/v | 0.5-4% | v/v | 0.75-3% | v/v | 0.75-2% | v/v |
| SB202190 | 10 | μM | 5-20 | μM | 5-15 | μM | 7.5-12.5 | μM |
| A83-01 | 500 | nM | 200-700 | nM | 400-600 | nM | 450-550 | nM |

Differentiation Medium
(Paneth Cell Enrichment
Medium)

| | | | | | | | | |
|---|---|---|---|---|---|---|---|---|
| Adv DMEM/F12 | 500 | mL | 500 | mL | 400-600 | mL | 450-550 | mL |
| Penicillin-Streptomycin (100x) | 5 | mL | 2.5-7.5 | mL | 2.5-7.5 | mL | 4-6 | mL |
| 200 mM L-glut (100x) | 5 | mL | 2.5-7.5 | mL | 2.5-7.5 | mL | 4-6 | mL |
| B27 (50x) | 10 | mL | 5-15 | mL | 5-15 | mL | 8-12 | mL |
| EGF (1000x) | 0.5 | mL | 0.25-0.75 | mL | 0.25-0.75 | mL | 0.4-0.6 | mL |
| Noggin (1000x) | 0.5 | mL | 0.25-0.75 | mL | 0.25-0.75 | mL | 0.4-0.6 | mL |
| 3 mM CHIR (1000x) | 0.5 | mL | 0.25-0.75 | mL | 0.25-0.75 | mL | 0.4-0.6 | mL |
| 5 mM DAPT (500x) | 1 | mL | 0.5-1.5 | mL | 0.5-1.5 | mL | 0.8-1.2 | mL |

Paneth Cell Enrichment
Medium (PC Medium)

| Adv DMEM/F12 | — | | | | | | | |
|---|---|---|---|---|---|---|---|---|
| B27 | 1x | | 0.5x-3x | | 0.5x-2x | | 0.5x-2x | |
| CHIR | 3 | μM | 1-4 | μM | 1-3 | μM | 1.5-2.5 | μM |
| Noggin | 100 | ng/mL | 25-175 | ng/mL | 50-150 | ng/mL | 75-125 | ng/mL |
| EGF | 100 | ng/mL | 25-175 | ng/mL | 50-150 | ng/mL | 75-125 | ng/mL |
| L-glut | 1% | v/v | 0.5-4% | v/v | 0.75-3% | v/v | 0.75-2% | v/v |
| Penicillin-Streptomycin | 1% | v/v | 0.5-4% | v/v | 0.75-3% | v/v | 0.75-2% | v/v |
| SB202190 | 10 | μM | 2-20 | μM | 5-15 | μM | 7.5-12.5 | μM |
| A83-01 | 500 | nM | 200-700 | nM | 400-600 | nM | 450-550 | nM |
| DAPT | 10 | μM | 2-20 | μM | 5-15 | μM | 7.5-12.5 | μM |

In various embodiments, the present invention provides pharmaceutical compositions including a pharmaceutically acceptable excipient along with enriched Paneth Cells of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In certain embodiments, the compounds of the present invention may contain one or more acidic functional groups and, thus, are capable of forming pharmaceutically acceptable salts with pharmaceutically acceptable bases. The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. These may include cations based on the alkali and alkaline earth metals such as sodium, lithium, potassium, calcium, magnesium and the like, as well as nontoxic ammonium, quaternary ammonium, and amine cations including, but not limited to ammonium, tetramethylanunonium, tetraethyl ammonium, methyl amine, dimethyl amine, trimethylamine, triethylamine, ethylamine, and the like (see, e.g., Berge S. M., et al. (1977) J. Pharm. Sci. 66, 1, which is incorporated herein by reference).

The term “pharmaceutically acceptable esters” refers to the relatively nontoxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. The term is further intended to include lower hydrocarbon groups capable of being solvated under physiological conditions, e.g., alkyl esters, methyl, ethyl and propyl esters.

As used herein, “pharmaceutically acceptable salts or prodrugs” are salts or prodrugs that are, within the scope of sound medical judgment, suitable for use in contact with the tissues of subject without undue toxicity, irritation, allergic response, and the like, commensurate with a reasonable benefit/risk ratio, and effective for their intended use.

The term “prodrug” refers to compounds that are rapidly transformed in vivo to yield the functionally active one or more peptides as disclosed herein or a mutant, variant, analog or derivative thereof. A thorough discussion is provided in T. Higachi and V. Stella, “Pro-drugs as Novel Delivery Systems,” Vol. 14 of the A. C. S. Symposium Series, and in Bioreversible Carriers in: Drug Design, ed. Edward B. Roche, American Pharmaceutical Association and Pergamon Press, 1987, both of which are hereby incorporated by reference. As used herein, a prodrug is a compound that, upon in vivo administration, is metabolized or otherwise converted to the biologically, pharmaceutically or therapeutically active form of the compound. A prodrug of the one or more peptides as disclosed herein or a mutant, variant, analog or derivative thereof can be designed to alter the metabolic stability or the transport characteristics of one or more peptides as disclosed herein or a mutant, variant, analog or derivative thereof, to mask side effects or toxicity, to improve the flavor of a compound or to alter other characteristics or properties of a compound. By virtue of knowledge of pharmacodynamic processes and drug metabolism in vivo, once a pharmaceutically active form of the one or more peptides as disclosed herein or a mutant, variant, analog or derivative thereof, those of skill in the pharmaceutical art generally can design prodrugs of the compound (see, e.g., Nogrady (1985) Medicinal Chemistry A Biochemical Approach, Oxford University Press, N. Y., pages 388-392). Conventional procedures for the selection and preparation of suitable prodrugs are described, for example, in “Design of Prodrugs,” ed. H. Bundgaard, Elsevier, 1985. Suitable examples of prodrugs include methyl, ethyl and glycerol esters of the corresponding acid.

### Kits

The present invention is also directed to a kit enriched for Paneth cells, a kit enriched for enterochromaffin cells, and a kit enriched for goblet cells, and a kit enriched for any combination of these cells. The kits are useful for practicing the inventive method of enriching for Paneth cells, enterochromaffin cells, and/or goblet cells. The kit is an assemblage of materials or components, including at least one of the inventive compositions.

The exact nature of the components configured in the inventive kit depends on its intended purpose. Instructions for use may be included in the kit. “Instructions for use” typically include a tangible expression describing the technique to be employed in using the components of the kit to effect a desired outcome. Optionally, the kit also contains other useful components, such as, diluents, buffers, cell media, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

iPSCs from a control individual and 2 CD patients were directed to HIOs and then dissociated to generate purified populations of epithelial only-HIOs (eHIOs). eHIOs were passaged weekly in proliferation medium (EGF, Noggin and CHIR99021) and the γ-secretase inhibitor, DAPT, was added to direct towards a Paneth cell fate. Flow cytometry, qPCR and immunocytochemistry were used to determine Paneth cell numbers, and gene and protein expression of various AMPs respectively.

iPSC-derived eHIOs from the control individual, which were used to develop this enrichment protocol, could be maintained for at least 4 months in proliferation medium. Flow cytometry analysis of the Paneth marker lysozyme revealed that the population of Paneth cells in eHIOs significantly increased from ~1% in proliferation media to ~28% upon treatment with DAPT. qPCR analysis demonstrated that DAPT treatment significantly increased the expression of the Paneth cell associated genes DEFA5, DEFA6, ITLN2, REG3A and PLA2G2A. Immunocytochemistry revealed that DAPT treated eHIOs were enriched for cells possessing granulated lysozyme, and also for additional AMPs such as DEFA5, ITLN2 and REG3A. The presence of the bacterial sensors TLR2, TLR5 and NOD2 were also detected in our Paneth cells. Finally, to confirm the applicability to CD patients, we applied our protocol and found the population of Paneth cells in CD eHIOs significantly increased from ~1% in proliferation media to ~10% upon treatment with DAPT, had significantly increased expression of the Paneth cell related genes and also were enriched for cells with granulated AMPs.

### Example 2

iPSCs from a control individual line (CS0003iCTR) were maintained in mTESR1 media on Matrigel coated plates.

iPSCs were directed, via a multistep protocol, to definitive endoderm, hindgut tissue and ultimately HIOs. As iPSC-derived HIOs are composed of both epithelial cells and mesenchymal cells, organoids were dissociated to a single cell suspension whereby epithelial cells were purified and cultured as epithelial-only HIOs (eHIOs). eHIOs are cultured in a proliferative medium containing EGF, noggin, and CHIR99021 and supplemented with SB202190 and A83-01. For differentiation to a Paneth cell fate, passaged eHIOs were cultured in proliferation medium for 2 days and subsequently cultured in a differentiation medium containing EGF, noggin and CHIR99021 with either IL22 or DAPT. After 12 days, eHIOs were processed for flow cytometry, qPCR and immunocytochemistry.

### Control Intestinal Organoids can be Directed to Paneth Cells

To determine if IL22 or CHIR99021 and DAPT directs eHIOs from a control iPSC line towards a Paneth cell fate eHIOs were cultured in proliferation media or in differentiation media with or without a forementioned factors and examined by flowcytometry, qPCR and immunocytochemistry

The addition of CHIR99021 and DAPT leads to an increase in the lysozyme+ cellular population. There is a significant increase in various Paneth cell related genes. The presence of various antimicrobial peptides were found via immunocytochemistry. The presence of various microbial sensors were observed via immunocytochemistry.

IPSCs from Crohn's Disease Patients can be Directed to Paneth Cells

To determine if treatment with CHIR99021 and DAPT directs eHIOs, derived from 2 Crohn's disease iPSC lines, towards a Paneth cell fate.

Two iPSC lines (CS508iIBD and CS748iIBD) were generated from Crohn's disease patients and directed to form eHIOs. They were cultured in proliferation media or in differentiation media with CHIR99021/DAPT and examined by flow cytometry, qPCR and immunocytochemistry.

This Paneth cell differentiation protocol was successfully carried out in 2 Crohn's iPSC lines.

### Example 3

This protocol outlines a method for the enrichment of Paneth cells (PCs) within human intestinal organoids (HIOs) derived from induced pluripotent stem cells (iPSCs). The process utilizes the small molecules CHIR99021 (CHIR) and DAPT to modulate Wnt/β-catenin and Notch signaling pathways, respectively, to promote PC differentiation and maturation. This protocol has successfully been verified in 4 different iPSC lines.

There are 2 crucial components in this methodology that need to be appreciated: (1) Human intestinal organoids need to be cultured for 30 days in vitro before they are sorted and grown as epithelial only-human intestinal organoids. This timepoint is ultimately crucial for Paneth cell enrichment; and (2) Epithelial only-human intestinal organoids are passaged weekly and must be passaged ≥10-15 times before the differentiation will occur.

Seeding and Culturing iPSCs for Differentiation

### Prepare the 24-Well Plate:

Coat the plate with Matrigel® (Corning 354234) by diluting 1 mg in 6 mL of base media.
Incubate at room temperature (RT) for 1 hour or store overnight at 4° C.
   Detach iPSCs:
Aspirate mTeSR™1 (STEMCELL Technologies 100-0483) media from the iPSC plate.
Add 1 mL ReLeSR™ to the well, allow it to sit for 30 seconds, and then aspirate.
Incubate at RT for 5 minutes until the cells appear brighter under the microscope.
   Collect iPSCs:
Gently wash the cells with 1 mL mTeSR™ using a P1000 pipette and transfer the suspension to a 15 mL tube, bringing the total volume to 12 mL.
Using a serological pipette, collect the cell suspension and transfer 0.5 mL into each well of the coated 24-well plate.

### Seed and Culture:

Gently shake the plate to distribute the cells uniformly.
Maintain the iPSCs in standard culture conditions to prepare them for differentiation into organoids.

### Beginning Definitive Endoderm and Hindgut Differentiation

Start differentiation when iPSCs reach 60-70% confluency.

### Definitive Endoderm Day 1:

Prepare RPMI (STEMCELL Technologies 36750) media supplemented with Wnt3a (25 ng/ml) and Activin A (100 ng/ml; R&D Systems 338-AC).
Add the media to the cells.

### Definitive Endoderm Day 2:

Expect some cell death and debris. Aspirate carefully to avoid disturbing the monolayer.
Prepare RPMI media supplemented with 0.2% FBS and Activin A (100 ng/mL) and add it to the cells.

### Definitive Endoderm Day 3:

Aspirate carefully to avoid disturbing the monolayer.
Prepare RPMI media supplemented with 2% FBS and Activin A (100 ng/mL) and add it to the cells.

### Hindgut Day 1 to Day 4:

Aspirate media carefully to avoid disturbing the monolayer.
Prepare Advanced DMEM/F12 (Gibco 12634-010) media supplemented with 2% FBS, FGF4 (500 ng/ml, R&D Systems, 235-F4), and CHIR (3 μM, Tocris, 4423), then add it to the cells.
Replace with fresh media daily.

### Transferring Hindgut Structures to Organoid Matrigel Bubbles

The day after Hindgut Day 4:
Without removing the media, gently scrape the cell monolayer off the plate using a P1000 or 5 mL pipette. Cells should detach as sheets.
Transfer the suspension into a 15 mL conical tube.
Gently triturate the suspension with a P1000 to break up large clumps (do not create a single-cell suspension).
Allow the structures to settle on ice for 15-20 minutes to form a loose pellet.
Carefully aspirate the media without disturbing the pellet.
Add Matrigel to the pellet, mix thoroughly with Matrigel, avoiding bubbles.
Pipette 50 μL Matrigel drops into the center of each well using a P200. Work quickly to prevent solidification.
Let drops solidify for 5-10 minutes.
Add 500 μL organoid media (refer to composition below) to each well.
Feed every other day and replate organoids every 5-7 days.
Grow the organoids for 30 days.

### Organoid Media Composition:

Organoid Medium

| | | |
|---|---|---|
| Adv DMEM/F12 | 500 | mL |
| Penicillin-Streptomycin (100x) | 5 | mL |
| 200 mM L-glut (100x) | 5 | mL |
| B27 (50x) | 10 | mL |
| EGF (1000x) (R&D, 236-RG | 0.5 | mL |
| Noggin (1000x) (R&D, 6057-NG) | 0.5 | mL |
| 2 mM CHIR (1000x) | 0.5 | mL |

### Sorting Epithelial Cells

### On Day 30:

### Harvest Organoids:

Use a P1000 pipette or a 5 mL serological pipette to gently scrape the Matrigel bubble off the plate.
Triturate the Matrigel bubble with media and transfer the mixture into a 15 ml conical tube.
Centrifuge at 1500 rpm for 3 minutes.
Carefully aspirate the supernatant, removing clean Matrigel but not the organoids.

### Wash Organoids:

Add 3 mL PBS to the pellet, triturate vigorously to break organoids out of the Matrigel, and repeat the wash twice.
Centrifuge at 1500 rpm for 3 minutes.
Aspirate the supernatant, ensuring only clean Matrigel is removed, leaving the organoids intact.

### Dissociate Organoids:

Resuspend the pellet in 1 mL TrypLE Select (Gibco 12563-029).
Incubate at 37° C. for 12 minutes.
Neutralize with 2 mL of 10% FBS/PBS solution.
Centrifuge at 1500 rpm for 3 minutes.

### Filter and Prepare for Sorting:

Carefully remove the supernatant and resuspend the pellet in 2 mL of 10% FBS/PBS solution.
Filter the suspension through a 30 μm filter into a new 15 ml conical tube.
Centrifuge at 1500 rpm for 3 minutes.
Aspirate the supernatant.
Add 50 μL EpCAM MicroBeads (Miltenyi Biotec 130-061-101), 50 μL Blocking Reagent (Miltenyi Biotec 130-059-901), and 500 μL 10% FBS/PBS solution.
Mix well and incubate on ice for 30 minutes.
Centrifuge at 1500 rpm for 3 minutes.
Aspirate the supernatant.
Resuspend the pellet in 1 mL of 10% FBS/PBS solution.
   Sort Cells on autoMACS Pro:
Proceed with sorting using the autoMACS Pro system.

### Post-Sort:

Spin down the sorted cells at 1500 rpm for 3 minutes.
Seed 50,000 cells per well in a 24 well plate.
Add 500 μL of eHIO media (refer to composition below).

eHIO Medium

| | | |
|---|---|---|
| Adv DMEM/F12 | 500 | mL |
| Penicillin-Streptomycin (100x) | 5 | mL |
| 200 mM L-glut (100x) | 5 | mL |
| B27 (50x) | 10 | mL |
| EGF (1000x) | 0.5 | mL |
| Noggin (1000x) | 0.5 | mL |
| 2 mM CHIR (1000x)* | 0.5 | mL |
| SB202190 (1000x)* | 0.5 | mL |
| A8301 (2000x)* | 0.25 | mL |
| Rock inhibitor (1000x)** | 0.5 | mL |

*CHIR, SB202190 and A8301: add fresh at each medium change.
**Rock inhibitor: add only during initial seeding.

Enrichment of Paneth Cells in eHIO culture

The epithelial organoids can be passaged weekly in eHIO medium, and to direct them toward a Paneth cell fate:
Seed 50,000 cells into a Matrigel bubble and maintain them in eHIO media for 2 days.
After 2 days, switch to differentiation media (refer to composition below).
On Day 7, replate the organoids and continue culture in differentiation media for an additional 5 days.
On Day 12, harvest the cells and proceed to analyze Paneth cell numbers and gene/protein expression using flow cytometry, qPCR, and immunocytochemistry.

Differentiation Medium

| | | |
|---|---|---|
| Adv DMEM/F12 | 500 | mL |
| Penicillin-Streptomycin (100x) | 5 | mL |
| 200 mM L-glut (100x) | 5 | mL |
| B27 (50x) | 10 | mL |
| EGF (1000x) | 0.5 | mL |
| Noggin (1000x) | 0.5 | mL |
| 3 mM CHIR (1000x) | 0.5 | mL |
| 5 mM DAPT (500x) | 1 | mL |

### Incorporation of Enriched Paneth Cell Organoids in Transwell Culture

Note: Organoids are polarized towards the lumen and accessing this aspect is crucial for determining responses to microbes and microbial components.
Transwells (0.33 cm²) were coated with Matrigel (prepared as described above) for 2 hrs at room temperature.
12 day eHIOs enriched for Paneth cells were dissociated as described
Use a P1000 pipette or a 5 mL serological pipette to gently scrape the Matrigel bubble off the plate.
Triturate the Matrigel bubble with media and transfer the mixture into a 15 mL conical tube.
Centrifuge at 1500 rpm for 3 minutes.
Carefully aspirate the supernatant, removing clean Matrigel but not the organoids
Add 3 mL PBS to the pellet, triturate vigorously to break organoids out of the Matrigel, and repeat the wash twice.
Centrifuge at 1500 rpm for 3 minutes. Aspirate the supernatant, ensuring only clean Matrigel is removed, leaving the organoids intact.
Resuspend the pellet in 1 mL TrypLE Select (Gibco 12563-029).
Incubate at 37° C. for 12 minutes.
Neutralize with 2 mL of 10% FBS/PBS solution.
Centrifuge at 1500 rpm for 3 minutes.
Resuspend cells at a density of 4×10⁶ cells/ml and seed 4×10⁵ cells per transwell
Cells are cultured for 24 hrs and can be treated with various cytokines/microbes/microbial ligands for 48 hrs.

### Example 4

### Cell Lines and Culture Conditions

The CS03iCTR-n1, CS2GW3iCTR-n1, and CS9EWPiCTR-n1 iPSC lines were obtained from the iPSC Core at Cedars-Sinai Medical Center (Los Angeles, CA, USA) and were derived from healthy control subjects. These lines were fully characterized for pluripotency markers and confirmed to be karyotypically normal. iPSCs were maintained in an undifferentiated state on Matrigel®-coated plates (Corning, 354234; Corning, NY, USA) in mTeSR™1 medium (STEMCELL Technologies, 85851; Vancouver, BC, Canada) under feeder-free conditions. Cells were passaged every 4-5 days using ReLeSR™ (STEMCELL Technologies, 100-0483; Vancouver, BC, Canada).

HIO Generation from iPSCs

The generation of human intestinal organoids (HIOs) from iPSCs was performed using a multistep protocol. Briefly, iPSCs were directed to definitive endoderm, followed by hindgut structures, and ultimately into organoids. All HIOs were cultured in medium containing CHIR99021 (2 μM; Tocris Bioscience, 4423; Bristol, UK), Noggin (100 ng/ml; R&D Systems, 6057-NG; Minneapolis, MN, USA), EGF (100 ng/ml; R&D Systems, 236-EG; Minneapolis, MN, USA), B-27 Supplement (1×; Gibco, 17504-044; Waltham, MA, USA), and DMEM/F12 (Gibco, 12634-010; Waltham, MA, USA), supplemented with penicillin/streptomycin and L-glutamine (5% v/v). All organoid cultures were embedded in standard Matrigel® Basement Membrane Matrix (Corning 354234), a non-growth factor-reduced formulation used consistently across all experiments. Organoids were passaged every 7-10 days and subjected to magnetic-activated cell sorting (MACS) after 30 days of differentiation.

### Generation of Purified Epithelial Cell Cultures

HIOs were removed from Matrigel and washed 3 times in Dulbecco's Phosphate-Buffered Saline (DPBS; Gibco, 14190-144; Waltham, MA, USA) and subsequently incubated in TrypLE™ Select (Gibco, 12563-029; Waltham, MA, USA) for 12 minutes until the organoids were completely dissociated to a single-cell suspension. Dissociated cells were incubated with EpCAM MicroBeads (Miltenyi Biotec, 130-061-101; Bergisch Gladbach, Germany) for 30 minutes at 4° C. EpCAM⁺ cells (eHIOs) were obtained via the autoMACS® Pro Separator (Miltenyi Biotec, Bergisch Gladbach, Germany). Following isolation, EpCAM⁺ epithelial cells were resuspended in Matrigel as single cells, where they self-organized and expanded to form epithelial-only HIOs (eHIOs). eHIOs were maintained in organoid medium supplemented with SB202190 (10 μM; Tocris Bioscience, 1264; Bristol, UK) and A83-01 (500 nM; Tocris Bioscience, A83-01; Bristol, UK).

### Enrichment of Paneth Cells

eHIOs were split once a week, and the medium was refreshed every three days. For passaging, eHIOs were removed from Matrigel, washed three times with DPBS (Gibco, 14190-144; Waltham, MA, USA), and incubated in TrypLE™ Select (Gibco, 12563-029; Waltham, MA, USA) for 12 minutes until fully dissociated into a single-cell suspension. Cells were then seeded into fresh Matrigel domes and maintained in eHIO expansion medium for 2 days. After 2 days, the culture medium was replaced with differentiation medium containing increased CHIR99021 (3 μM; Tocris Bioscience, 4423; Bristol, UK), IL-22 (2 ng/ml; R&D Systems, 782IL; Minneapolis, MN, USA) and DAPT (10 μM; R&D Systems, 2634/10; Minneapolis, MN, USA) to promote Paneth cell differentiation. Organoids were maintained in this differentiation medium for 5 days. Subsequently, the expanded eHIOs were replated without enzymatic dissociation and cultured for an additional 5 days in the same differentiation medium. On Day 12 of the differentiation protocol, cells were harvested and analyzed for Paneth cell enrichment. To assess Paneth cell antimicrobial function at the transcriptional level, eHIOs were stimulated with IL-22 (10 ng/ml; R&D Systems, 782IL; Minneapolis, MN, USA) for 72 or 120 hours, starting on days 10 and 12 of the differentiation protocol, respectively.

Media formulations used for HIO and eHIO culture and Paneth cell enrichment. This table lists all components, final concentrations, suppliers, and catalog numbers for each media condition used in the study, including HIO maintenance medium, epithelial-only HIO (eHIO) expansion medium, and the modified ENC(3) and ENC(3)+DAPT formulations. Stage-specific notes are provided to enable full replication of the differentiation and enrichment protocol

| | | | Final | | Supplier/ | |
|---|---|---|---|---|---|---|
| Media | Duration | Component | Conc. | | Catalog No. | Notes |
| 1. Organoid | Passaged | Adv | — | | Gibco, | Base medium |
| Medium | every 7-10 | DMEM/F12 | | | 12634-010 | |
| (HIO | days per to | B-27 | 1x | | Gibco, | Added at all stages |
| Medium) | MACS sort. | Supplement | | | 17504-044 | |
| Base | (Culture | CHIR99021 | 2 | μM | Tocris, 4423 | Wnt activation; |
| formulation | duration: 30 | | | | | maintained through |
| used pre- | days) | | | | | HIO culture |
| MACS + | | Noggin | 100 | ng/mL | R&D Systems, | BMP inhibition |
| A2: F14 sort | | | | | 6057-NG | |
| | | EGF | 100 | ng/mL | R&D Systems, | Supports epithelial |
| | | | | | 236-EG | growth |
| | | L-glutamine | 1% | v/v | Gibco | Metabolic support |
| | | Penicillin- | 1% | v/v | Gibco | Antimicrobial |
| | | Streptomycin | | | | |
| 2. Epithelial- | Passaged | HIO Medium | — | | — | Base formulation |
| only | every 7 days | SB202190 | 10 | μM | Tocris, 1264 | p38 inhibitor; add fresh |
| Organoid | as part of cell | | | | | at each medium change |
| Medium | culture | A83-01 | 500 | nM | Tocris, A83-01 | TGF-β inhibitor; add |
| (eHIO | maintenance. | | | | | fresh at each medium |
| Medium) | Medium | | | | | change |
| Base | refreshed | | | | | |
| formulation | every 2 days | | | | | |
| used post- | | | | | | |
| MACS sort | | | | | | |
| 3. Paneth | 1. Culture single | HIO Medium | — | | — | Base formulation |
| Cell | epithelial cells in | (as above) | | | | |
| Enrichment | matrigel domes for | CHIR99021 | 3 | μM | Tocris, 4423 | Wnt activator; add fresh |
| Medium (PC | 2 days in eHIO | | | | | at each medium change |
| Medium) | medium; | DAPT | 10 | μM | R&D Systems, | Notch inhibitor; add |
| Base | 2. On 3^{rd} day | | | | 2634/10 | fresh at each medium |

| | | |
|---|---|---|
| formulation | switch to PC | change; drives Paneth |
| used for | medium for 5 days. | cell differentiation |
| Paneth Cell | 3. Split developed | |
| differentiation | organoid structure | |
| | into new matrigel | |
| | domes from 1 to 2 | |
| | parts and culture | |
| | in PC medium | |
| | for another 5 | |
| | days (no single | |
| | cell dissociation); | |
| | Medium refreshed | |
| | every 2 days. | |
| | (Culture duration: | |
| | 12 days; 2 days | |
| | eHIO medium + 10 | |
| | days PC Medium) | |

Quantitative Polymerase Chain Reaction (qPCR)

RNA was extracted from eHIOs using the RNeasy® Mini Kit (Qiagen, 74106; Hilden, Germany). Complementary DNA (cDNA) was synthesized from 500 ng of RNA using the High-Capacity cDNA Reverse Transcription Kit (Applied Biosystems™, 4368814; Foster City, CA, USA). Quantitative real-time PCR was performed using SYBR™ Select Master Mix (Applied Biosystems™, 4472908; Foster City, CA, USA) on a Bio-Rad CFX384 Real-Time System (Bio-Rad Laboratories, Hercules, CA, USA). Primer sequences are listed below.

| | | SEQ | | SEQ |
|---|---|---|---|---|
| | | ID | | ID |
| Gene Name | Forward Primer | NO | Reverse Primer | NO |
| EF1A | TATTGGATTGCCACACGGCT | 1 | AAAGCGACCCAAAGGTGGA | 2 |
| | | | T | |
| LYZ | TTTCTGTTACGGTCCAGGGC | 3 | ACACATCCAGTTTGCTAGGC | 4 |
| | | | T | |
| DEFA5 | TGGGGAAGACAACCAGGAC | 5 | TTCGGCAATAGCAGGTGG | 6 |
| DEFA6 | GAGGATGCAAGCTCAAGTCT | 7 | TGCAATGGCAAGTGAAAGC | 8 |
| | TAG | | | |
| REG3A/G | ACTGCTATGCCTTGTTTTTGT | 9 | CTACTCCACTCCCAACCTTC | 10 |
| | C | | TC | |
| SPLA2G2A | CGCACTCAGTTATGGCTTCTA | 11 | GTGATTCTGCTCCCCGAG | 12 |
| | C | | | |
| ITLN2 | CTCCTTTTCTTCCCTGCCTAG | 13 | TCTGGTAGACAACACCATTC | 14 |
| | | | TTG | |
| PRSS2 | CCCTCATCAGCGAACAGTG | 15 | CAGGATGTCATTGTCCAGAG | 16 |
| | | | TC | |
| TLR2 | CGGCGTTCTCTCAGGTGA | 17 | TGGCATTGTCCAGTGCTTC | 18 |
| TLR5 | TGCTACTGACAACGTGGCTT | 19 | ACATCTGAGGCTCCGACATC | 20 |
| | C | | | |
| NOD2 | GTGGAGAACATGCTGGACCT | 21 | GCCAATGGGACTGGTAATTC | 22 |

### Immunocytochemistry and Microscopy

eHIOs were fixed in 4% paraformaldehyde (Electron Microscopy Sciences, 15714-5; Hatfield, PA, USA), transferred to 30% sucrose overnight at 4° C., embedded in Tissue-Tek® O.C.T. Compound (Sakura Finetek, 4583; Torrance, CA, USA), and cryosectioned at 8 μm thickness. Sections were blocked with 10% normal donkey serum in 0.5% Triton X-100 and incubated overnight at 4° C. with primary antibodies (Table 2). The following day, sections were rinsed and incubated with Alexa Fluor 488 (1:1000; Invitrogen, A21202; Carlsbad, CA, USA), Alexa Fluor 594 (1:1000; Invitrogen, A32758; Carlsbad, CA, USA) or Alexa Fluor 647 (1:1000; Invitrogen, A21447; Carlsbad, CA, USA) for 1 hour at room temperature, followed by DAPI counterstaining (1:5000; Invitrogen, H3570; Carlsbad, CA, USA). Slides were imaging using Nikon Eclipse Ti-A1R HD.

TABLE 2

| | Host | | Catalog | |
|---|---|---|---|---|
| Antigen | Species | Manufacturer | number | Dilution |
| Lysozyme | Mouse | Novus Biologicals | NB100-63062 | 1/500 |
| Lysozyme | Rabbit | Invitrogen | PA5-16668 | 1/250 |
| NOD2 | Mouse | Novus Biologicals | NB100-524 | 1/100 |
| DEFA5 | Mouse | Novus Biologicals | NB110-60002 | 1/1250 |
| ITLN2 | Mouse | R&D Systems | MAB8004 | 1/100 |
| Reg3A | Mouse | R&D Systems | MAB5965 | 1/200 |
| E-cadherin | Goat | R&D Systems | AF648 | 1/1000 |
| TLR2 | Rabbit | Novus Biologicals | NB100-56720 | 1/250 |
| TLR5 | Mouse | Novus Biologicals | NBP2-24787 | 1/250 |

### Organoid Epithelial Cell Monolayer

Paneth cell-enriched eHIOs were dissociated into single cells using TrypLE™ Select (Gibco, 12563-029; Waltham, MA, USA) and seeded onto 6.5 mm Transwell® inserts (STEMCELL Technologies, 38024; Vancouver, BC, Canada) pre-coated with Matrigel® (Corning, 354234; Corning, NY, USA) at a density of 4.0×10⁵ cells per well. Cells were cultured in differentiation medium for 24 hours to allow monolayer formation and stabilization prior to stimulation.

### Enzyme-Linked Immunosorbent Assay (ELISA)

Medium from transwell inserts were collected after exposure to LPS (100 μg/mL; Sigma-Aldrich, L2880; St. Louis, MO, USA), Pam3CSK4 (10 μg/mL; Novus Biologicals, NBP2-25297; Centennial, CO, USA), and MDP (100 μg/mL; InvivoGen, tlrl-mdp; San Diego, CA, USA) for 30 minutes. Human lysozyme ELISA (Novus Biologicals, NBP2-60511; Centennial, CO, USA) was performed according to the manufacturer's instructions. Absorbance at 450 nm and 570 nm was measured using a SpectraMax M3 microplate reader (Molecular Devices, San Jose, CA, USA).

### Flow Cytometry

eHIOs were removed from Matrigel, washed three times with DPBS (Gibco, 14190-144; Waltham, MA, USA), and incubated in TrypLE™ Select (Gibco, 12563-029; Waltham, MA, USA) for 12 minutes at 37° C. to generate a single-cell suspension. Dissociated cells were passed through a 70 μm mini cell strainer (pluriSelect, 43-10070-40; Leipzig, Germany) to remove aggregates and debris. Cells were then fixed in Flow Cytometry Fixation Buffer (R&D Systems, FC004; Minneapolis, MN, USA) and permeabilized using Flow Cytometry Permeabilization/Wash Buffer (R&D Systems, FC005; Minneapolis, MN, USA), following the manufacturer's protocol. Permeabilized cells were incubated with unconjugated primary antibodies:anti-Lysozyme (1:100; Novus Biologicals, NB100-63062; Centennial, CO, USA) and anti-DEFA5 (1:2000; Novus Biologicals, NB110-60002; Centennial, CO, USA) for 30 minutes at 4° C. After three washes, cells were incubated with Alexa Fluor 488-conjugated secondary antibody (1:100; Invitrogen, A21202; Carlsbad, CA, USA) for 30 minutes at 4° C. in the dark. Samples were analyzed using a BD LSR Fortessa™ Cell Analyzer (BD Biosciences, San Jose, CA, USA), and data were analyzed with FlowJo software (BD Biosciences, San Jose, CA, USA).

### Transmission Electron Microscopy

Organoid samples were fixed in 2.5% glutaraldehyde and 2% formaldehyde in 0.1 M sodium cacodylate at 4° C. After fixation, samples were dehydrated and embedded in Epon 812 (Polysciences). The ultrathin sections were stained with 1% uranyl acetate and 3% lead citrate. Ultimately, the images were taken using HT7700 transmission electron microscope (HITACHI) at 1,200× direct magnification.

Bacterial Isolation from Crohn's Disease Intestinal Resections

Intestinal tissue specimens were collected aseptically from patients with Crohn's disease undergoing ileocolonic resections. Immediately following surgical excision, tissues were placed in sterile containers and transferred from the operating room to a biosafety cabinet for processing within 1 h of resection. Tissues were rinsed with sterile DPBS (Gibco, 14190-144; Waltham, MA, USA) until all visible surface blood was removed. Sutured ends were excised, and the bowel was opened longitudinally. Mucosal scrapings were obtained, yielding 200-500 μL of mucosal material per specimen, which was transferred into sterile microcentrifuge tubes. Mucosal scrapings were immediately placed in an anaerobic chamber (Coy Laboratory Products, 602000; Grass Lake, MI, USA) maintained at 90% N₂, 5% CO₂, and 5% H₂. Samples were homogenized in 1 mL of reduced sterile DPBS and an aliquot (100 μL) of the homogenate was inoculated into pre-reduced 9.9 mL brain heart infusion (BHI; Sigma-Aldrich, B21202; St. Louis, MO, USA) and 9.9 mL Gifu Anaerobic Medium (GAM; HiMedia, M189; Mumbai, India). Cultures were incubated anaerobically at 37° C. for ≥12 h. Overnight cultures from both media were pooled, and bacterial suspensions were normalized to an optical density at 600 nm (OD₆₀₀). TW was treated with the pooled bacterial suspension at a final concentration of OD₆₀₀=0.5 for 30 min, after which the media was collected for analysis.

### Proteomics Sample Digest

Samples were digested using the SP3 protocol. Briefly, 20 μg of protein was brought to 60 μL with lysis buffer containing 6 M urea, 1 M ammonium bicarbonate, and 5% SDS (Thermo Fisher, 20230; Waltham, MA, USA). Proteins were reduced with 16.8 μL of 200 mM dithiothreitol (DTT; Sigma-Aldrich, D9779; St. Louis, MO, USA) for 30 min at 37° C. with shaking at 300 rpm, then alkylated with 21.2 μL of 400 mM iodoacetamide (IAA; Sigma-Aldrich, I1149; St. Louis, MO, USA) at room temperature for 30 min in the dark. The volume was brought to 160 μL with Tris-HCl pH 8 (Thermo Fisher, AM9856; Waltham, MA, USA), and 5 μL of bead suspension (10:1 mass ratio of beads to protein; 1:1 mixture of hydrophilic/hydrophobic beads; Cytiva, 29152179; Marlborough, MA, USA) was added and vortexed. Samples were adjusted to 70% acetonitrile (ACN; Sigma-Aldrich, 34851; St. Louis, MO, USA) v/v and incubated for 18 min. Solvent was removed on-magnet, and samples were washed with 2×80% ethanol (EtOH; Sigma-Aldrich, 459844; St. Louis, MO, USA) and 2×ACN (200 μL each). After complete solvent removal, samples were resuspended in 50 mM Tris-HCl pH 8 with 10 mM CaCl₂ (Sigma-Aldrich, C1016; St. Louis, MO, USA) and digested with trypsin (1:20 enzyme:protein; Promega, V5111; Madison, WI, USA). Samples were bath-sonicated for 5 min, then incubated for 18 h at 37° C. with shaking at 1200 rpm. After digestion, samples were removed from beads and brought to 0.1% formic acid (FA; Sigma-Aldrich, F8775; St. Louis, MO, USA) and 2% DMSO (Sigma-Aldrich, 276855; St. Louis, MO, USA) for LC-MS/MS injection.

### LC-MS/MS Analysis

Approximately 500 ng of peptides from digested samples were analyzed on a Thermo Orbitrap Astral mass spectrometer (Thermo Fisher, A12345; Waltham, MA, USA) coupled to a NeoVanquish LC (Thermo Fisher, NLC123; Waltham, MA, USA). A blank injection was included after every three samples to assess carryover. Peptides were separated using a 24 min gradient with solvent A (0.1% formic acid in water; Sigma-Aldrich, F8875; St. Louis, MO, USA) and solvent B (80% acetonitrile, 0.1% formic acid; Sigma-Aldrich, 34851; St. Louis, MO, USA) at 1.2 μL/min. The gradient was: 0 min, 4% B; 2 min, 9% B; 13 min, 25% B; 17 min, 35% B. LC was performed in direct injection mode using a PepSep C18 column (150 μm ID×15 cm, 1.5 μm; Bruker, 100367; Billerica, MA, USA) coupled to a nano source (Thermo EasySpray, ES081; Waltham, MA, USA). MS data were acquired in data-independent acquisition (DIA) mode from 380-980 Da with 240 k Orbitrap resolution, 10 ms maximum injection time for MS1, and 4 ms maximum injection time for MS2 with 4 Th windows.

### Data Analysis

MS raw data files were searched against UniProt human reviewed protein sequence entries (accessed April 2023) using DIA-NN (v 1.8.1) in library free mode with default parameters. Based on recent comparisons with library-based approaches, DIA-NN in library-free mode has been found to produce results that are comparable or better than those of experimental library-based searches while being freely available and was hence chosen for the analysis of all data (PMID: 36609502). The output protein group matrix from DIA-NN was used to perform downstream analysis using MetaboAnalyst 6.0.

### Gene Ontology Enrichment Analysis

Proteins identified in the secretome were filtered for statistical significance using a p-value<0.05 and false discovery rate (FDR)<0.05. The filtered list of proteins was submitted to STRING (version 12.0; https://string-db.org) for functional enrichment analysis of Gene Ontology (GO) Biological Processes, using Homo sapiens as the reference species. STRING calculates enrichment using a hypergeometric test and corrects for multiple comparisons with the Benjamini-Hochberg procedure. Enrichment results are reported as STRING-derived FDR values.

### Statistical Analysis

All data are presented as mean±standard error of the mean (SEM). Statistical analyses were performed using GraphPad Prism version 9.2.0 (GraphPad Software, San Diego, CA, USA). Comparisons among multiple groups were conducted using two-way analysis of variance (two-way ANOVA) followed by appropriate post hoc tests. Unpaired or paired t-tests were used for comparisons between two groups, as appropriate. A p-value<0.05 was considered statistically significant.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

What is claimed is:

1. A method of differentiating epithelial human intestinal organoids into Paneth Cells and enriching the Paneth Cells in eHIO culture, comprising:
seeding about 25,000-75,000 human epithelial intestinal cells into a MATRIGEL bubble and maintaining the human epithelial intestinal cells in eHIO expansion medium for about 1-3 days;
replacing the eHIO medium with differentiation medium and culturing for about 4-6 days;
replating the human epithelial intestinal cells and continuing to culture in differentiation medium for an additional 4-6 days thereby generating a cell culture enriched with Paneth cells; and
harvesting cells from the cell culture enriched with Paneth cells.

2. The method of claim 1, wherein the method comprises:
seeding about 50,000 human epithelial intestinal cells into a Matrigel bubble and maintaining the human epithelial intestinal cells in eHIO expansion medium for about 2 days;
replacing the eHIO expansion medium with differentiation medium and culturing for about 5 days;
replating the human epithelial intestinal cells and continuing culture in differentiation medium for an additional 5 days, thereby generating a cell culture enriched with Paneth cells; and
harvesting cells from the cell culture enriched with Paneth cells.

3. The method of claim 1, further comprising analyzing Paneth cell numbers and gene expression and/or protein expression using flow cytometry, qPCR, immunocytochemistry, or combinations thereof.

4. The method of claim 1, wherein
the Paneth cells have an increased mRNA expression of one or more genes selected from the group consisting of DEFA5, DEFA6, PLA2G2A, REG3A, and ITLN2, as compared to undifferentiated eHIOs, or
wherein the Paneth cells have an increased mRNA expression of DEFA5, DEFA6, PLA2G2A, and REG3A, as compared to undifferentiated eHIOs, or
wherein the Paneth cells have an increased mRNA expression of HD5, HD6, PLAZAG2A, and REG3A, or have the presence of lysozyme, HD5, REG3A, or both, as compared to undifferentiated eHIOs, or
wherein the Paneth cells are LYZ⁺ and DEFA5⁺.

5. The method of claim 1, wherein the cell culture comprising Paneth cells show a presence of electron dense granules via immunocytochemistry.

6. A method of differentiating epithelial human intestinal organoids into enterochromaffin cells, goblet cells or both and enriching the enterochromaffin cells, goblet cells, or both in eHIO culture, comprising:
seeding about 25,000-75,000 human epithelial intestinal cells into a MATRIGEL bubble and maintaining the human epithelial intestinal cells in eHIO medium for about 1-3 days;
replacing the eHIO medium with differentiation medium and culturing for about 4-6 days;
replating the human epithelial intestinal cells and continuing to culture in differentiation medium for an additional 4-6 days thereby generating a cell culture enriched with enterochromaffin cells, goblet cells, or both; and
harvesting cells from the cell culture enriched with enterochromaffin cells, goblet cells, or both.

7. The method of claim 6, wherein
the enterochromaffin cells, goblet cells, or both have increased mRNA expression of CHGA and MUC2, as compared to undifferentiated eHIOs, or
wherein the enterochromaffin cells are CHGA⁺ and serotonin⁺.

8. The method of claim 1, wherein eHIO medium comprises Adv DMEM/F12, L-glut, B27, EGF, Noggin, CHIR, SB202190, A8301, and optionally, Rock inhibitor, and Penicillin-Streptomycin.

9. The method of claim 8, wherein the CHIR is at a concentration of at least 3 mM.

10. The method of claim 1, wherein differentiation medium comprises Adv DMEM/F12, Penicillin-Streptomycin, 200 mM L-glut, B27, EGF, Noggin, CHIR, and DAPT.

11. The method of claim 10, wherein the CHIR is at a concentration of at least 3 mM.

12. The method of claim 1, wherein the human epithelial intestinal cells are differentiated from human intestinal organoids (HIOs) that have been cultured for about 15-50 days before being sorted and grown as epithelial only-human intestinal organoids.

13. The method of claim 1, wherein the human epithelial intestinal cells are differentiated from human intestinal organoids (HIOs) that have been cultured for at least 30 days before being sorted and grown as epithelial only-human intestinal organoids.

14. The method of claim 1, wherein the human epithelial intestinal cells are from epithelial only-human intestinal organoids (eHIOs) that have been passaged for at least 5 times.

15. The method of claim 1, wherein the human epithelial intestinal cells are from epithelial only-human intestinal organoids (eHIOs) that have been passaged for about 10-15 times.

16. The method of claim 1, wherein the epithelial human intestinal cells are obtained by:
removing HIOs from MATRIGEL and washing the HIOs;
dissociating the HIOs into dissociated cells;
separating and isolating EpCAM+ cells;
resuspending EpCAM+ cells in MATRIGEL as single cells, wherein the EpCAM+ cells self-organize and expand to form epithelial-only HIOs (eHIOs);
maintaining the eHIOs in organoid medium supplemented with SB202190 and A83-01; and
dissociating the eHIOs into single cell suspension.

17. A method of incorporating enriched human Paneth cell organoids in a transwell culture, comprising:
dissociating epithelial human intestinal organoids (eHIOs) enriched for human Paneth cells by:
removing a MATRIGEL bubble off a plate comprising eHIOs enriched for human Paneth cells;
breaking up the MATRIGEL bubble with medium and transferring the mixture into a tube;
centrifuging at about 1000-2000 rpm for about 1-5 minutes thereby generating supernatant and pellet;
aspirating the supernatant to remove the MATRIGEL;
adding about 1-5 mL PBS to the pellet, breaking organoids out of the MATRIGEL, and repeating 1-3 times;
centrifuging at about 1000-2000 rpm for about 1-5 minutes thereby generating supernatant and pellet;
removing the supernatant to remove MATRIGEL;
resuspending the pellet in about 0.5-1.5 mL TrypLE Select;
incubating at about 34-40° C. for about 10-14 minutes;
neutralizing with about 1-3 mL of about 12-14% FBS/PBS solution;
centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

18. The method of claim 17, wherein the method comprises:
dissociating eHIOs enriched for human Paneth cells by:
using a pipette to scrape a MATRIGEL bubble off a plate comprising eHIOs enriched for human Paneth cells;
triturating the MATRIGEL bubble with medium and transferring the mixture into a 15 ml conical tube;
centrifuging at about 1500 rpm for about 3 minutes thereby generating supernatant and pellet;
aspirating the supernatant to remove MATRIGEL but not the organoids;
adding about 3 mL PBS to the pellet, triturating to break organoids out of the MATRIGEL, and repeat the wash twice;
centrifuging at about 1500 rpm for about 3 minutes thereby generating supernatant and pellet;
aspirating the supernatant, ensuring only clean Matrigel is removed, leaving the organoids intact;
resuspending the pellet in about 1 mL TrypLE Select;
incubating at about 37° C. for about 12 minutes;
neutralizing with about 2 mL of about 10% FBS/PBS solution;
centrifuging at about 1500 rpm for about 3 minutes;
resuspending cells at a density of about 4×10⁶ cells/ml and seed about 4×10⁵ cells per transwell; and
culturing the cells for about 24 hrs.

19. A method of incorporating enriched human into enterochromaffin cells, goblet cells or both in a transwell culture, comprising:
dissociating epithelial human intestinal organoids (eHIOs) enriched for enterochromaffin cells, goblet cells or both by:
removing a MATRIGEL bubble off a plate comprising eHIOs enriched for human into enterochromaffin cells, goblet cells or both;
breaking up the MATRIGEL bubble with medium and transferring the mixture into a tube;
centrifuging at about 1000-2000 rpm for about 1-5 minutes thereby generating supernatant and pellet;
aspirating the supernatant to remove the MATRIGEL;
adding about 1-5 mL PBS to the pellet, breaking enterochromaffin cells, goblet cells or both out of the MATRIGEL, and repeating 1-3 times;
centrifuging at about 1000-2000 rpm for about 1-5 minutes thereby generating supernatant and pellet;
removing the supernatant to remove MATRIGEL;
resuspending the pellet in about 0.5-1.5 mL TrypLE Select;
incubating at about 34-40° C. for about 10-14 minutes;
neutralizing with about 1-3 mL of about 12-14% FBS/PBS solution;
centrifuging at about 1000-2000 rpm for about 1-5 minutes;
resuspending cells at a density of about 4×10⁵ to 4×10⁷ cells/ml and seed about 4×10⁴ to 4×10⁶ cells per transwell; and
culturing the cells for about 12-36 hours.

20. Human Paneth cell organoids obtained by the method of claim 1.

21. A human Paneth cell organoid model, comprising: human Paneth cell organoids of claim 20.

22. A transwell culture, comprising: the human Paneth cell organoid model of claim 21.

23. An enterochromaffin cells, goblet cells or both obtained by the method of claim 6.

24. An enterochromaffin cells model, goblet cell model or both, comprising enterochromaffin cells, goblet cells or both of claim 23.

25. A transwell culture obtained by the method of claim 17.

26. A transwell culture obtained by the method of claim 19.
